(19) United States
(12) Patent Application Publication
(10) Pub. No.: US 2014/0024620 A1
(43) Pub. Date: Jan. 23, 2014

(54) Methods for Inhibiting Cell Proliferation in EGFR-Driven Cancers

(71) Applicants: David C. Dalgarno, Brookline, MA (US); William C. Shakespeare, Southborough, MA (US); Xiaotian Zhu, Newton, MA (US); Victor M. Rivera, Arlington, MA (US); Juan J. Miret, Shrewsbury, MA (US)

(72) Inventors: David C. Dalgarno, Brookline, MA (US); William C. Shakespeare, Southborough, MA (US); Xiaotian Zhu, Newton, MA (US); Victor M. Rivera, Arlington, MA (US); Juan J. Miret, Shrewsbury, MA (US)

(73) Assignee: ARIAD PHARMACEUTICALS, INC., Cambridge, MA (US)

(21) Appl. No.: 13/878,744

(22) PCT Filed: Oct. 14, 2011
(86) PCT No.: PCT/US11/56457

§ 371 Date: Sep. 5, 2013

Related U.S. Application Data
(60) Provisional application No. 61/393,291, filed on Oct. 14, 2010.

(51) Int. Cl. C07F 9/6558; C07D 401/12; C07F 9/6568; C07F 9/6561

(52) U.S. Cl. CPC C07F 9/65583; C07F 9/6561; C07D 401/12; C07F 9/6568
USPC 514/81; 435/375; 514/86; 514/252.18

(57) ABSTRACT

The invention features a method for treating patients who have an EGFR-driven cancer, which is, or has become, refractory to a tyrosine kinase inhibitor, such as eriotinib and gefitinib, by administering a compound of formula (I) to the patient. The invention also features treating EGFR-driven cancers having an EGFR mutation identified herein.

### BACKGROUND OF THE INVENTION

This invention relates to pharmaceutical compositions and methods for inhibiting the proliferation of cells and for the treatment of certain cancers.

Specific genetic lesions that drive the proliferation of cancer cells, such as those causing activation of certain tyrosine kinases, render some cancers highly sensitive to therapeutic agents that inhibit the kinase. However, the efficacy of such agents is often limited by the development of mutations in the target kinase domain that confer resistance by reducing inhibitor binding.

For example, the ABL kinase inhibitor imatinib has revolutionized treatment for patients with chronic myeloid leukemia (CML), whose disease is driven by an activated BCR-ABL fusion oncoprotein. Over time, however, development of mutations in the ABL kinase domain confers resistance in a substantial proportion of patients. The second-generation ABL inhibitors dasatinib and nilotinib, by virtue of being more potent inhibitors of ABL, demonstrate superior efficacy and are able to overcome much of the mutation-based resistance exhibited by imatinib.

More recently genetic lesions in the epidermal growth factor receptor (EGFR) have been identified that show a similar pattern of sensitivity to first-generation inhibitors and susceptibility to mutation-based resistance. Activating mutations in EGFR have been identified in 10-20% of patients with NSCLC, and the EGFR kinase inhibitors gefitinib and erlotinib have demonstrated activity in these patients.

Activating mutations for EGFR, which can take the form of small deletions or point mutations in the kinase domain, have been cataloged and described at length in the scientific literature. See e.g., Sharma, Nat. Rev. Cancer 7:169 (2007) (exon 19 mutations characterized by in-frame deletions of amino-acids 747 account for 45% of mutations, exon 21 mutations resulting in L858R substitutions account for 40-45% of mutations, and the remaining 10% of mutations involve exon 18 and 20); Sordella et al., Science 305:1163 (2004); and Mulloy et al., Cancer Res. 67:2325 (2007).

However, the clinical efficacy of gefitinib and erlotinib is ultimately limited by the development of resistance, such as by mutation in the EGFR kinase domain gatekeeper residue (T790M), which occurs in 50% of patients.

There is a clear need for new methods to inhibit cells with EGFR mutations like T790M that confer resistance to current EGFR tyrosine kinase inhibitor (“TKI”) products. New therapies for treating cancers associated with such mutations would be of profound benefit.

### SUMMARY OF THE INVENTION

The invention features a class of compounds having the structure of formula (I), below:

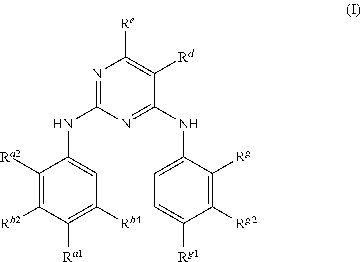

in which

R^{d} is H, C₁₋₄ alkyl, C₁₋₄ alkoxy, or halo; and R^{e} is H or NH₂, or R^{d} and R^{e}, together with the pyrimidine ring atoms to which they are attached, form a 5- or 6-membered ring containing one, two or three heteroatoms, independently selected from N, S and O, wherein the 5- or 6-membered ring is substituted by R^{h};

R^{h} is H, C₁₋₄ alkyl, or halo;

R^{a2} is H, C₁₋₆ alkoxy, C₃₋₆ alkenyloxy, or C₃₋₆ cycloalkyloxy;

R^{g} is —P(O)(R^{3A})(R^{3B}), —S(O)N(R^{3C})(R^{3D}), —S(O)₂R^{3E}, —OC(O)N(R^{3F})(R^{3G}), —NR^{3H}C(O)OR^{3I}, a 5 or 6 member heterocyclic ring comprising 1, 2, 3 or 4 N atoms, or combined with R^{g2} forms a 5- to 7-member heterocyclic ring, wherein each of R^{3A}, R^{3B}, R^{3C}, R^{3D}, R^{3E}, R^{3F}, R^{3G}, R^{3H}, and R^{3I} is, independently, selected from H, alkyl, alkenyl, alkynyl, cycloalkyl, cycloalkenyl, cycloalkynyl, and heteroalkyl, or R^{3A} and R^{3B}, or R^{3C} and R^{3D}, or R^{3F} and R^{3G}, together with the atoms to which they are attached, combine to form a 5- or 6-membered heterocyclic ring which is unsubstituted or substituted;

R^{g2} is H, F, C₁₋₄ alkyl, or, R^{g2} and R^{g} together with the atoms to which they are attached form a 5- to 7-member heterocyclic ring comprising 1-3 hetero atoms independently selected from P, N, O and S, the heterocyclic ring being unsubstituted or substituted;

R^{g1} is H, F, or a 5 or 6 member heterocyclic ring comprising 1 or 2 N atoms, the heterocyclic ring being unsubstituted or substituted;

R^{b2} is H, F, or is a 5 or 6 member heterocyclic ring containing 1, 2 or 3 N or O atoms, the heterocyclic ring being unsubstituted or substituted;

R^{b4} is H, F, C₁₋₆ alkoxy, C₃₋₆ alkenyloxy, or C₃₋₆ cycloalkyloxy, —OC(O)N(R^{5A})(R^{5B}), —NR^{5C}C(O)OR^{5D}; a 5 or 6 member heterocyclic ring comprising 1, 2 or 3 N or O atoms, the heterocyclic ring being unsubstituted or substituted, or, R^{b4} and R^{a1} together with the atoms to which they are attached form a 6 member heterocyclic ring comprising 1, 2 or 3 N or O atoms which is unsubstituted or substituted;

each of R^{5A}, R^{5B}, R^{5C}, and R^{5D} is, independently, selected from H, alkyl, alkenyl, alkynyl, and heteroalkyl, or R^{5A} and R^{5B}, together with the atoms to which they are attached, combine to form a 5- or 6-membered heterocyclic ring which is unsubstituted or substituted;

R^{a1} combines with R^{b4} to form a 6 member heterocyclic ring, or is H, halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or

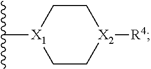

each Y is, independently, a bond, —O—, —S— or —NR¹—;

each occurrence of R¹ and R² is, independently, selected from H, alkyl, alkenyl, alkynyl, cycloalkyl, cycloalkenyl, cycloalkynyl, aryl, heteroalkyl, heterocyclic and heteroaryl;

each of X₁ and X₂ is, independently, selected from CH and N; and

R⁴ is selected from alkyl, alkenyl, alkynyl, cycloalkyl, cycloalkenyl, cycloalkynyl, aryl, heteroalkyl, heterocyclic and heteroaryl. In some embodiments the R⁴ moiety bears one or more substituents as discussed further below.

In certain embodiments, R^{d} may in addition be cyclopropyl.

One subclass of compounds of particular current interest for use in practicing the method of this invention are those compounds of formula (I) in which R^{a2} is C₁₋₆ alkoxy, C₃₋₆ alkenyloxy, or C₃₋₆ cycloalkyloxy, and R^{g} is —P(O)(R^{3A})(R^{3B}), —S(O)N(R^{3C})(R^{3D}), —S(O)₂R^{3E}, and pharmaceutically acceptable salts of such compounds.

The compounds of formula (I), its subclasses and its various embodiments (as are discussed in further detail below) are active inhibitors of EGFR mutants, including EGFR proteins (a) with an activating mutation such as L858R or delE746_A750, (b) with a resistance-conferring mutation such as T790M, and (c) with both types of mutations. That is significant because while cancers characterized by an activating mutation in EGFR might be treatable with erlotinib or gefitinib, that is not the case if the EGFR bears a resistance-conferring mutation, whether alone or in combination with an (otherwise) activating mutation. The inability of existing EGFR inhibitors like erlotinib and gefitinib to effectively inhibit the resistant EGFR mutants or the cancers with which they are associated leaves patients with a terrible lack of treatment options. Because the compounds disclosed herein inhibit cases where the prior TKI's cannot, they are of interest as new treatment options.

Moreoever, the compounds of formula (I) that preferentially inhibit the mutant EGFR over wild-type EGFR are of particular interest, especially when the preference is at least 10-fold, even more at 100-fold, and of greatest interest at 500-fold or more. That preferential inhibition can be readily measured with conventional methods, such as biochemical determinations of relative IC50 values of the compound for the wild-type and mutant EGFRs, by measurement of its relative growth inhibitory effect on cells transformed with the respective forms of EGFR, etc. preferential inhibition of the mutant EGFR over wildtype EGFR helps reduce the risk

Accordingly, this invention provides a method for treating an EGFR-driven cancer in a subject by administering to the subject a therapeutically effective amount of compound of formula (I), or a pharmaceutically acceptable salt thereof. The method is of particular significance for subjects whose cancer is refractory to erlotinib or gefitinib, or whose cancer is characterized by the presence of the T790M EGFR mutation or other mutation associated with resistance to erlotinib or gefitinib, alone or in combination with an activating mutation.

The invention further provides a method for treating an EGFR-driven cancer in a subject including the steps of (a) providing a subject having an EGFR-driven cancer characterized by the presence of a mutation in epidermal growth factor receptor kinase (EGFR), and (b) administering to the subject a therapeutically effective amount of compound of formula (I), or a pharmaceutically acceptable salt thereof. For example, the EGFR-driven cancer can be characterized by the presence of one or more mutations selected from: (i) L858R, (ii) T790M, (iii) both L858R and T790M, (iv) delE746_A750, (v) both delE746_A750 and T790M, and any other EGFR mutations described herein.

In the above methods, the EGFR-driven cancer can be a non-small cell lung cancer (NSCLS); glioblastoma; pancreatic cancer; head and neck cancer (e.g., squamous cell carcinoma); breast cancer; colorectal cancer; epithelial cancer; ovarian cancer; prostate cancer; an adenocarcinoma, or any EGFR-driven cancer.

In a related aspect, the invention features a method of inhibiting the proliferation of a cell expressing an EGFR mutant, the method including contacting the cell with a compound of formula (I), or a pharmaceutically acceptable salt thereof, in an amount sufficient to inhibit the proliferation. For example, the cell can be characterized by the presence of one or more mutations in EGFR selected from: (i) L858R, (ii) T790M, (iii) both L858R and T790M, (iv) delE746_A750, (v) both delE746_A750 and T790M, and any other EGFR mutations described herein. In certain embodiments, the cell is a cancer cell (e.g., a cell from a non-small cell lung cancer (NSCLS); glioblastoma; pancreatic cancer; head and neck cancer (e.g., squamous cell carcinoma); breast cancer; colorectal cancer; epithelial cancer; ovarian cancer; prostate cancer; an adenocarcinoma, or any other EGFR expressing cancer described herein).

The invention further features a method of treating an EGFR-driven cancer refractory to a first kinase inhibitor selected from erlotinib, gefitinib, and pharmaceutically acceptable salts thereof, in a subject by administering to the subject a compound of formula (I), or a pharmaceutically acceptable salt thereof, in an amount sufficient to treat the cancer.

In any of the above methods for inhibiting cell proliferation or treating a subject with an EGFR-driven cancer, the compound of formula (I) is described by the formula:

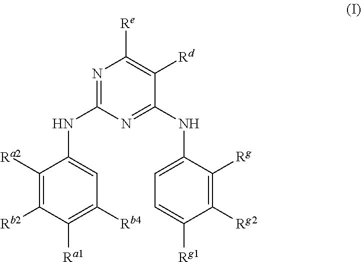

In formula (I), R^{d} is H, C₁₋₄ alkyl, C₁₋₄ alkoxy, or halo; and R^{e} is H or NH₂; or R^{d} and R^{e}, together with the pyrimidine ring atoms to which they are attached, form a 5- or 6-membered ring containing 1, 2 or 3 heteroatoms, independently selected from N, S and O, wherein the 5- or 6-membered ring is substituted by R^{h}; R^{h} is H, C₁₋₄ alkyl, or halo; R^{a2} is H, alkoxy, C₃₋₆ alkenyloxy, or C₃₋₆ cycloalkyloxy; R^{g} is —P(O)(R^{3A})(R^{3B}), —S(O)N(R^{3C})(R^{3D}), —S(O)₂R^{3E}, —OC(O)N(R^{3F})(R^{3G}), —NR^{3H}C(O)OR^{3I}, a 5 or 6 member heterocyclic ring comprising 1, 2, 3 or 4 N atoms, or combined with R^{g2} forms a 5- to 7-member heterocyclic ring; each of R^{3A}, R^{3B}, R^{3C}, R^{3D}, R^{3E}, R^{3F}, R^{3G}, R^{3H}, and R^{3I} is, independently, selected from H, alkyl, alkenyl, alkynyl, cycloalkyl, cycloalkenyl, cycloalkynyl, and heteroalkyl, or R^{3A} and R^{3B}, or R^{3C} and R^{3D}, or R^{3F} and R^{3G}, together with the atoms to which they are attached, combine to form a 5- or 6-membered heterocyclic ring which is unsubstituted or substituted; R^{g2} is H, F, C₁₋₄ alkyl, or, R^{g2} and R^{g} together with the atoms to which they are attached form a 5- to 7-member heterocyclic ring comprising 1-3 hetero atoms independently selected from P, N, O and S, the heterocyclic ring being unsubstituted or substituted; R^{g1} is H, F, or a 5 or 6 member heterocyclic ring comprising 1 or 2 N atoms, the heterocyclic ring being unsubstituted or substituted; R^{b2} is H, F, or is a 5 or 6 member heterocyclic ring containing 1, 2 or 3 N or O atoms, the heterocyclic ring being unsubstituted or substituted; R^{b4} is H, F, C₁₋₆ alkoxy, C₃₋₆ alkenyloxy, or C₃₋₆ cycloalkyloxy, —OC(O)N(R^{5A})(R^{5B}), or —NR^{5C}C(O)OR^{5D}; a 5 or 6 member heterocyclic ring comprising 1, 2 or 3 N or O atoms, the heterocyclic ring being unsubstituted or substituted, or, R^{b4} and R^{al} together with the atoms to which they are attached form a 6 member heterocyclic ring comprising 1, 2 or 3 N or O atoms which is unsubstituted or substituted; each of R^{5A}, R^{5B}, R^{5C}, and R^{5D} is, independently, selected from H, alkyl, alkenyl, alkynyl, and heteroalkyl, or R^{5A} and R^{5B}, together with the atoms to which they are attached, combine to form a 5- or 6-membered heterocyclic ring which is unsubstituted or substituted; R^{a1} combines with R^{b4} to form a 6 member heterocyclic ring, or is H, halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or

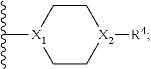

each Y is, independently, a bond, —O—, —S— or —NR¹—; each occurrence of R¹ and R² is, independently, selected from H, alkyl, alkenyl, alkynyl, cycloalkyl, cycloalkenyl, cycloalkynyl, aryl, heteroalkyl, heterocyclic and heteroaryl; each of X₁ and X₂ is, independently, selected from CH and N; and R⁴ is selected from alkyl, alkenyl, alkynyl, cycloalkyl, cycloalkenyl, cycloalkynyl, aryl, heteroalkyl, heterocyclic and heteroaryl.

In formula (I), for any of R^{a2}, R^{d}, R^{h}, R¹, R², R⁴, R^{3A}, R^{3B}, R^{3C}, R^{3D}, R^{3E}, R^{3F}, R^{3G}, R^{3H}, and R^{3I} selected from C₁₋₆ alkoxy, C₃₋₆ alkenyloxy, C₃₋₆ cycloalkyloxy, alkyl, alkenyl, alkynyl, cycloalkyl, cycloalkenyl, cycloalkynyl, aryl, heteroalkyl, heterocyclic, and heteroaryl, the substituent is substituted or unsubstituted. In particular embodiments, the compound of formula (I) used in practicing the method of this invention is further described by formula (IIa) or formula (IIb):

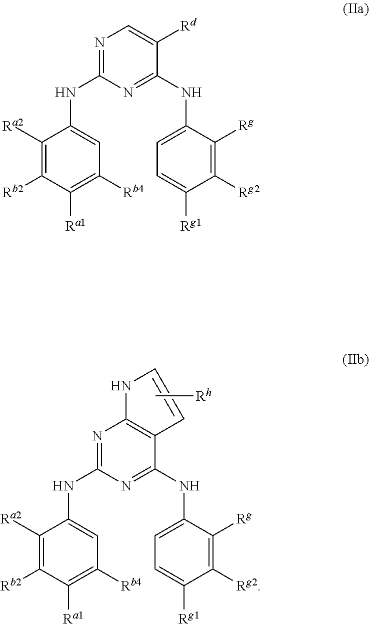

In formulas (IIa) and (IIb), R^{a1}; R^{a2}; R^{b2}; R^{b4}; R^{g}; R^{g1}; R^{g2}; R^{d}; and R^{h} are as defined above.

In certain embodiments, the compound used in practicing the method of this invention is a compound of formula (I), (IIa), or (IIb) in which R^{g1}, R^{g2}, R^{b2} and R^{b4} are H or F.

In another embodiment, the compound used is a compound of formula (IIa) in which R^{d} is Cl, F or CF₃.

In another embodiment, the compound used is a compound of formula (I), (IIa), or (IIb), in which R^{a1} is —OMe. In others, R^{a1} is YP(═O)(YR¹)(YR²), —NR¹SO₂R², —S(O)ᵣR², —SO₂NR¹R² or —NR¹SO₂NR¹R².

In another embodiment, the compound used is a compound of formula (I), (IIa), or (IIb), in which R^{a2} is C₁₋₆ alkoxy, C₃₋₆ alkenyloxy, or C₃₋₆ cycloalkyloxy; and in a particular embodiment thereof, R^{a2} is methoxy.

In another embodiment, the compound is of formula (I), (IIa), or (IIb), and R^{g} is —P(O)(R^{3A})(R^{3B}) or —S(O)₂R^{3E}. In one embodiment of those compounds, R^{a2} is C₁₋₆ alkoxy, C₃₋₆ alkenyloxy, or C₃₋₆ cycloalkyloxy.

In another embodiment, the compound is of formula (I), (IIa), or (IIb), and R^{a1} is a 5- or 6-membered heterocyclic ring comprising one or two heteroatoms selected from N and O, the ring being unsubstituted or substituted with an alkyl group.

In some embodiments, the method uses a compound of formula (I) which can further be described by formula (III):

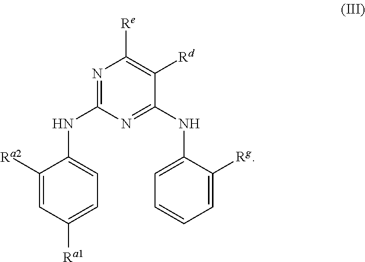

In formula (III), R^{a2} is alkoxy; R^{g} is —P(O)(R^{3A})(R^{3B}), —S(O)N(R^{3C})(R^{3D}), or —S(O)₂R^{3E}; each of R^{3A}, R^{3B}, R^{3C}, R^{3D}, and R^{3E} is, independently, H or C₁₋₇ alkyl, or R^{3A} and R^{3B}, or R^{3C} and R^{3D}, together with the atoms to which they are attached, combine to form a 5- or 6-membered heterocyclic ring which is unsubstituted or substituted; R^{d} is H, C₁₋₄ alkyl, C₁₋₄ alkoxy, or halo; and R^{e} is H or NH₂; or R^{d} and R^{e}, together with the pyrimidine ring atoms to which they are attached, form a 5- or 6-membered ring containing one or two heteroatoms, independently N, S or O, and the 5- or 6-membered ring is substituted by R^{h}; R^{h} is H, C₁₋₄ alkyl, or halo; R^{a1} is halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or

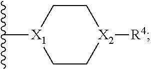

each Y is, independently, a bond, —O—, —S— or —NR¹—; each occurrence of R¹ and R² is, independently, H, alkyl, alkenyl, alkynyl, cycloalkyl, cycloalkenyl, cycloalkynyl, aryl, heteroalkyl, heterocyclic or heteroaryl; each X₁ and X₂ is, independently, CH or N; and R⁴ is alkyl, alkenyl, alkynyl, cycloalkyl, cycloalkenyl, cycloalkynyl, aryl, heteroalkyl, heterocyclic or heteroaryl.

Certain embodiments use a compound of formula (III) further described by formula (IVa) or formula (IVb):

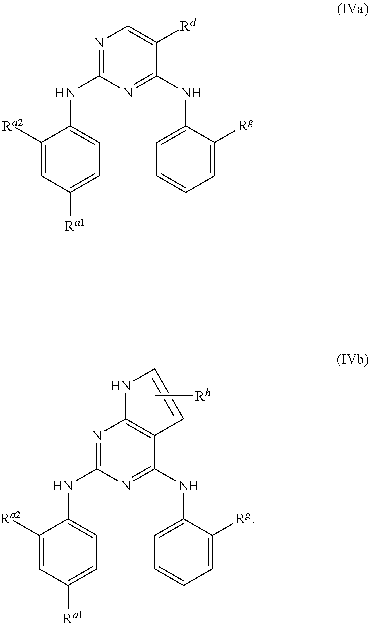

In formulas (IVa) and (IVb), R^{a2}; R^{g}; R^{d}; R^{h}; and R^{a1} are as defined above for formula (III).

In certain embodiments, the compound used is of any of formulas (III), (IVa) and (IVb), and R^{a2} is a methoxy, ethoxy, or propoxy group.

In some embodiments, the compounds used is of formula (III) and (IVa), and R^{d} is selected from Cl, F, CF₃, and cyclopropyl;

In still other embodiments, the compound of formula (III) is further described by any of formulas (Va)-(Vd):

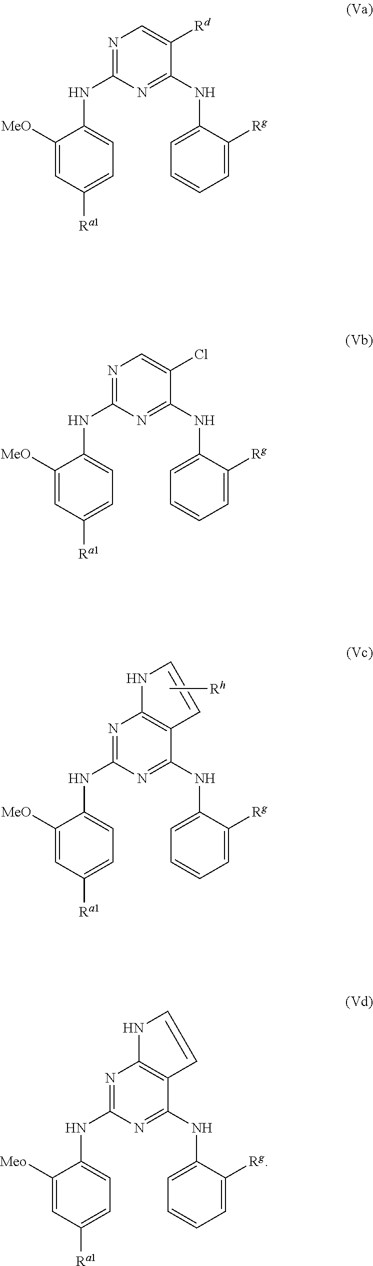

In formulas (Va)-(Vd), R^{g}; R^{d}; R^{h}; and R^{a1} are as defined above for formula (III).

In particular embodiments, the compounds used are of any of the above formulas, and R^{g} is —P(O)(CH₃)₂, —P(O)(CH₂CH₃)₂, or —S(O)₂(CH(CH₃)₂).

In particular embodiments, the compounds of any of the above formulas, have as an R^{a1} moiety:

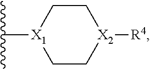

wherein X₁, X₂, and R⁴ are as defined above for formula (III). For example, R^{a1} can be selected from any of the following groups:

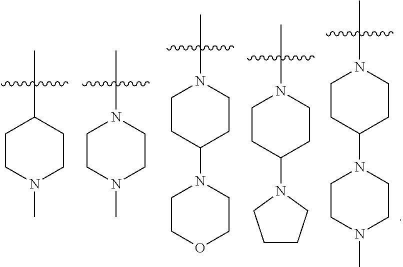

R^{a1} can also be selected from groups with additional substitution, or with less substitution, as illustrated by the following additional exemplary R^{a1} choices:

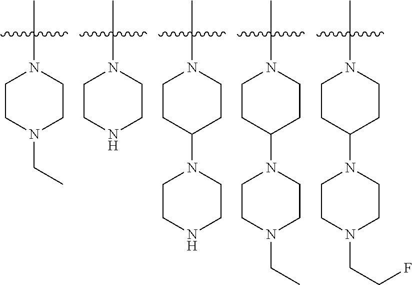

and by numerous other examples herein which illustrate a wider range of R^{a1} choices.

In particular embodiments, the compound of formula (I) is further described by one of formulas (VIa)-(VIf):

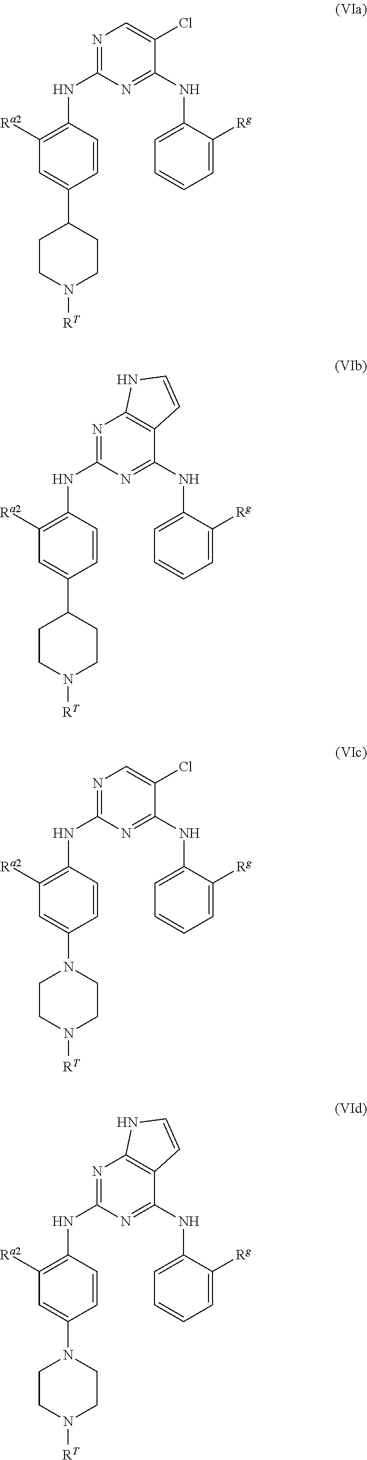

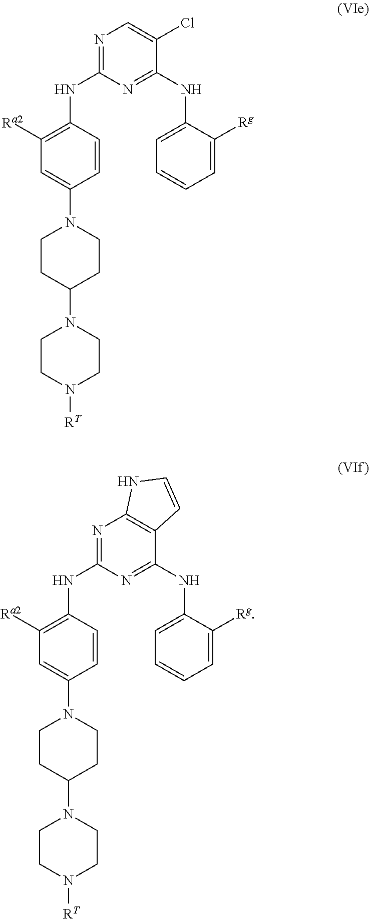

In formulas (VIa)-(VIf), R^{a2} is a methoxy, ethoxy, or propoxy group; R^{g} is —P(O)(CH₃)₂, —P(O)(CH₂CH₃)₂, or —S(O)₂(CH(CH₃)₂); and, in certain embodiments, R^{T} is methyl. In other such embodiments of formulas (VIa)-(VIf), R^{T} is H, acyl or C1-C4 alkyl which may be substituted or unsubstituted, e.g., —CH₃, —CH₂CH₃ or —CH₂CH₂OH.

In any of the above formulas, the compound can be in the form of a free base or a pharmaceutically acceptable salt thereof.

The compounds of formula (I) include those described in PCT Publication Nos. WO2009/143389, WO 2006/021454, WO 2006/021457, and WO2009/126515, each of which is incorporated herein by reference.

### DEFINITIONS

The clinical response to the methods of the invention can be graded according to the response evaluation criteria in solid tumors (RECIST) guidelines (see Eur. J. Cancer 45:228 (2009)) that define when cancer patients improve (“respond”), stay the same (“stabilize”), or worsen (“progression”) during treatments. A complete response is characterized by: (i) disappearance of all target lesions; and (ii) any pathological lymph nodes (whether target or non-target) must have reduction in short axis to <10 mm. A partial response is characterized by: (i) at least a 30% decrease in the sum of diameters of target lesions, taking as reference the baseline sum diameters. A progressive disease is characterized by (i) at least a 5%, 10%, or 20% increase in the sum of diameters of target lesions, taking as reference the smallest sum on study (this includes the baseline sum if that is the smallest on study); or (ii) the appearance of one or more new lesions.

The term “administration” or “administering” refers to a method of giving a dosage of a pharmaceutical composition to a mammal, where the method is, e.g., oral, intravenous, intraperitoneal, intraarterial, or intramuscular. The preferred method of administration can vary depending on various factors, e.g., the components of the pharmaceutical composition, site of the potential or actual disease and severity of disease. While compounds of formula I will generally be administered perorally, other routes of administration can be useful in carrying out the methods of the invention.

By “EGFR-driven cancer” is meant a cancer characterized by a mutation in an EGFR gene that alters the biological activity of an EGFR nucleic acid molecule or polypeptide, including the specific mutations noted herein. EGFR-driven cancers can arise in any tissue, including brain, blood, connective tissue, liver, mouth, muscle, spleen, stomach, testis, and trachea. EGFR-driven cancers include non-small cell lung cancer (NSCLS), including one or more of squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; adenocarcinomas; and including cancers which are EGFR mediated.

An “EGFR mutant” or “mutant” includes one or more deletions, substitutions, or additions in the amino acid or nucleotide sequences of EGFR protein, or EGFR coding sequence. The EGFR mutant can also include one or more deletions, substitutions, or additions, or a fragment thereof, as long as the mutant retains or increases tyrosine kinase activity, compared to wild type EGFR. In particular EGFR mutations, kinase or phosphorylation activity can be increased (e.g., by at least 5%, 10%, 15%, 20%, 30%, 40%, 50%, 60%, 70%, 80%, 90%, or even 100%), as compared to wild type EGFR. Particular EGFR mutants are described herein, where mutations are provided relative to the position of an amino acid in human EGFR, as described in the sequence provided in NOBI GenBank Reference Sequence: NP_{—}005219.2.

As used herein, the term “inhibiting the proliferation of a cell expressing an EGFR mutant” refers to measurably slowing, stopping, or reversing the growth rate of the EGFR-expressing cells in vitro or in vivo. Desirably, a slowing of the growth rate is by at least 10%, 20%, 30%, 50%, or even 70%, as determined using a suitable assay for determination of cell growth rates (e.g., a cell growth assay described herein). The EGFR mutant can be any EGFR mutant described herein.

As used herein, the term “refractory” refers to a cancer which is progressive despite application of a particular therapy. The cancer can be refractory either from the initial administration of the therapy; or become refractory over time in response to the therapy. “Resistance” to a drug refers to reduced sensitivity to that drug as determined by any scientifically valid comparative analysis.

The term “sequence identity” is meant the shared identity between two or more nucleic acid sequence, or two or more amino acid sequences, expressed in the terms of the identity between the sequences. Sequence identity can be measured in terms of percentage identity; the higher the percentage, the more identical the sequences are. Homologs or orthologs of nucleic acid or amino acid sequences possess a relatively high degree of sequence identity when aligned using standard methods. Methods of alignment of sequences for comparison are well known in the art. Various programs and alignment algorithms are described in: Smith and Watermann, Adv. Appl. Math. 2:482 (1981); Needleman and Wunsch, J. Mol. Biol. 48:443 (1970); Pearson and Lipman, Proc. Natl. Acad. Sci. U.S.A. 85:2444 (1988); Corpet et al., Nuc. Acid Res. 16:10881 (1988); Huang et al., Computer Appls. in the Biosciences 8:155 (1992); and Pearson et al., Meth. Mol. Biol. 24:307 (1994). Altschul et al. (J. Mol. Biol. 215:403 (1990)) presents a detailed consideration of sequence alignment methods and homology calculations. The NCBI Basic Local Alignment Search Tool (BLAST) (Altschul et al., J. Mol. Biol. 215:403 (1990)) is available from several sources, including the National Center for Biological Information (NCBI) website, for use in connection with the sequence analysis programs blastp, blastn, blastx, tblastn, and tblastx. Additional information can be found at the NCBI website. BLASTN is used to compare nucleic acid sequences, while BLASTP is used to compare amino acid sequences. To compare two nucleic acid sequences, the option can be set as follows: −i is set to a file containing the first nucleic acid sequence to be compared; −j is set to a file containing the second nucleic acid sequence to be compared; −p is set to blastn; −o is set to any desired file name; −q is set to −1; −r is set to 2; and all other options are left at their default setting. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 30, 35, 40, 45, 50, 60, 70, 80, 90, 100, 150, 200, 250, 300, 350, or 400 consecutive nucleotides or amino acid residues from a sequence set forth in an identified sequence), followed by multiplying the resulting value by 100. One indication that two nucleic acid molecules are closely related is that the two molecules hybridize to each other under stringent conditions. Stringent conditions are sequence-dependent and are different under different environmental parameters. Nucleic acid molecules that hybridize under stringent conditions to an EGFR gene sequence typically hybridize to a probe based on either an entire EGFR gene or selected portions of the gene (e.g., the kinase domain or a segment of the gene that contains the mutated codons described herein), under conditions described above.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

The term “alkyl” refers to linear, branched, cyclic, and polycyclic non aromatic hydrocarbon groups, which may be substituted or unsubstituted. Unless otherwise specified, “alkyl” groups contain one to eight, and preferably one to six carbon atoms. Lower alkyl refers to alkyl groups containing 1 to 6 carbon atoms. Examples of alkyl include, without limitation, methyl, ethyl, n-propyl, isopropyl, cyclopropyl, butyl, isobutyl, sec-butyl, tert-butyl, cyclobutyl, pentyl, isopentyl tert-pentyl, cyclopentyl, hexyl, isohexyl, cyclohexyl, and n-heptyl, among others. Exemplary substituted alkyl groups include, without limitation, haloalkyl groups (e.g., fluoromethyl, difluoromethyl, trifluoromethyl, 2-fluoroethyl, 3-fluoropropyl), hydroxymethyl, 2-hydroxyethyl, 3-hydroxypropyl, benzyl, substituted benzyl, and phenethyl, among others.

The term “alkoxy” refers to a subset of alkyl in which an alkyl group as defined above with the indicated number of carbons attached through an oxygen bridge, —O-alkyl, wherein the alkyl group contains 1 to 8 carbons atoms and is substituted or unsubstituted. Exemplary alkoxy groups include, without limitation, methoxy, ethoxy, n-propoxy, i-propoxy, t-butoxy, n-butoxy, s-pentoxy, —OCF₃, and —O-cyclopropyl.

The term “haloalkyl” refers to a subset of alkyl in which an alkyl group as defined above having one or more hydrogen atoms of the alkyl substituted with a halogen atom. Exemplary haloalkyl groups include, without limitation, trifluoromethyl, trichloromethyl, pentafluoroethyl and the like.

The term “alkenyl” refers to a branched or unbranched hydrocarbon group containing one or more double bonds and having from 2 to 8 carbon atoms. An alkenyl may optionally include monocyclic or polycyclic rings, in which each ring desirably has from three to six members. The alkenyl group may be substituted or unsubstituted. Alkenyl groups include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

The term “alkynyl” refers to a branched or unbranched hydrocarbon group containing one or more triple bonds and having from 2 to 8 carbon atoms. The alkynyl group may be substituted or unsubstituted. Alkynyls include, without limitation, ethynyl, 1-propynyl, 2-propynyl, 1-butynyl, 2-butynyl, and 3-butynyl.

The term “cycloalkyl” refers to cyclic or polycyclic hydrocarbon groups of from 3 to 13 carbon atoms, any of which is saturated. Cycloalkyl groups may be substituted or unsubstituted. Exemplary cycloalkyl groups include, without limitation, cyclopropyl, norbornyl, [2.2.2]bicyclooctane, and [4.4.0]bicyclodecane, and the like, which, as in the case of other alkyl moieties, may optionally be substituted.

The term “cycloalkenyl” refers to cyclic or polycyclic hydrocarbon groups of from 3 to 13 carbon atoms, preferably from 5 to 8 carbon atoms, containing one or more double bonds. Cycloalkenyl groups may be substituted or unsubstituted. Exemplary cycloalkenyl groups include, without limitation, cyclopentenyl, cyclohexenyl, and cyclooctenyl.

The term “cycloalkynyl” refers to cyclic or polycyclic hydrocarbon groups of from 5 to 13 carbon atoms containing one or more triple bonds. Cycloalkynyl groups may be substituted or unsubstituted.

The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Examples of heteroalkyls include, without limitation, polyethers, such as methoxymethyl and ethoxyethyl.

As used herein, “heterocyclic ring” and “heterocyclyl” refer to non-aromatic ring systems having five to fourteen ring atoms in which one or more ring carbons, preferably one to four, are each replaced by a heteroatom such as N, O, S, or P, which may be used alone or as part of a larger moiety as in “heterocyclyl-alkyl” (a heterocyclyl-substituted C₁₋₆ alkyl), “heterocyclyl-alkoxy” (a heterocyclyl-substituted C₁₋₆ alkoxy), or “heterocycloxy-alkyl” (a heterocycloxy-substituted C₁₋₆ alkyl), and includes aralkyl, aralkoxy, and aryloxyalkyl groups. Heterocyclic rings may be substituted or unsubstituted and may include one, two, or three fused or unfused ring systems. Desirably, the heterocyclic ring is a 5- to 7-membered monocyclic or 7- to 14-membered bicyclic heterocyclic ring consisting of 2 to 6 carbon atoms and 1, 2, 3, or 4 heteroatoms independently selected from N, O, and S and including any bicyclic group in which any of the above-defined heterocyclic rings is fused to a benzene ring. Exemplary heterocyclic rings include, without limitation, 3-1H-benzimidazol-2-one, (1-substituted)-2-oxo-benzimidazol-3-yl, 2-tetrahydrofuranyl, 3-tetrahydrofuranyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. A heterocyclyl group can include two or more of the ring systems listed above. Heterocyclic rings include those in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring.

The term “aryl” used alone or as part of a larger moiety as in “aralkyl” (an aryl-substituted C₁₋₆ alkyl), “aralkoxy” (an aryl-substituted C₁₋₆ alkoxy), or “aryloxyalkyl” (an aryloxy-substituted C₁₋₆ alkyl), refers to aromatic monocyclic or polycyclic ring groups having six to fourteen ring atoms, such as phenyl, 1-naphthyl, 2-naphthyl, 1-anthracyl, and 2-anthracyl and includes aralkyl, aralkoxy, and aryloxyalkyl groups. An “aryl” ring may be substituted or unsubstituted. The term “aryl” includes fused polycyclic aromatic ring systems in which an aromatic ring is fused to one or more rings. Non-limiting examples of aryl groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro, 1-naphthyl, 2-naphthyl, 1-anthracyl, and 2-anthracyl. Also included within the scope of the term “aryl”, as it is used herein, is a group in which an aromatic ring is fused to one or more non-aromatic rings, such as in a indanyl, phenanthridinyl, or tetrahydronaphthyl, where the radical or point of attachment is on the aromatic ring.

The term “heteroaryl” as used herein refers to stable heterocyclic, and polyheterocyclic aromatic moieties having 5-14 ring atoms. Heteroaryl groups may be substituted or unsubstituted and include both monocyclic and polycyclic ring systems. Examples of typical heteroaryl rings include 5-membered monocyclic rings, such as thienyl, pyrrolyl, imidazolyl, pyrazolyl, furyl, isothiazolyl, furazanyl, isoxazolyl, and thiazolyl; 6-membered monocyclic rings, such as pyridyl, pyrazinyl, pyrimidinyl, pyridazinyl, and triazinyl; and polycyclic heterocyclic rings, such as benzo[b]thienyl, naphtho[2,3-b]thienyl, thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxalinyl, quinazolinyl, benzothiazole, benzimidazole, tetrahydroquinoline cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl (see e.g. Katritzky, Handbook of Heterocyclic Chemistry). Exemplary heteroaryl rings include, without limitation, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, and benzoisoxazolyl. Heteroaryl groups further include a group in which a heteroaromatic ring is fused to one or more aromatic or nonaromatic rings where the radical or point of attachment is on the heteroaromatic ring, such as tetrahydroquinoline, tetrahydroisoquinoline, and pyrido[3,4-d]pyrimidinyl, imidazo[1,2-a]pyrimidyl, imidazo[1,2-a]pyrazinyl, imidazo[1,2-a]pyiridinyl, imidazo[1,2-c]pyrimidyl, pyrazolo[1,5-a][1,3,5]-triazinyl, pyrazolo[1,5-c]pyrimidyl, imidazo[1,2-b]pyridazinyl, imidazo[1,5-a]pyrimidyl, pyrazolo[1,5-b][1,2,4]triazine, quinolyl, isoquinolyl, quinoxalyl, imidazotriazinyl, pyrrolo[2,3-d]pyrimidyl, triazolopyrimidyl, and pyridopyrazinyl.

An aryl group or heteroaryl group may contain one or more substituents. Exemplary substituents for an aryl or heteroaryl group include halogen (F, Cl, Br or I), alkyl, alkenyl, alkynyl, heteroalkyl, —NO₂, —CN, —R^{A}, —OR^{B}, —S(O)ᵣR^{B}, (wherein r is 0, 1 or 2), —SO₂NR^{A}R^{B}, —NR^{A}R^{B}, —O—NR^{A}R^{B}, —NR^{A}—NR^{A}R^{B}, —(CO)YR^{B}, —O(CO)YR^{B}, —NR^{A}(CO)YR^{B}, —S(CO)YR^{B}, —NR^{A}C(═S)YR^{B}, —OC(═S)YR^{B}, —C(═S)YR^{B}, —YC(═NR^{A})YR^{B}, —YC(═N—OR^{A})YR^{B}, —YC(═N—NR^{A}R^{B})YR^{B}, —COCOR^{B}, —COMCOR^{B} (where M is a C₁₋₆ alkyl group), —YP(O)(YR^{C})(YR^{C}), —P(O)(R^{C})₂, —Si(R^{C})₃, —NR^{A}SO₂R^{B}, and —NR^{A}SO₂NR^{A}R^{B}, wherein each occurrence of Y is, independently, —O—, —S—, —NR^{A}—, or a chemical bond (i.e., —(CO)YR^{B} thus encompasses —C(═O)R^{B}, —C(═O)OR^{B}, and —C(═O)NR^{A}R^{B}).

R^{C} is selected from alkyl, alkenyl, alkynyl, cycloalkyl, cycloalkenyl, cycloalkynyl, aryl, heteroaryl, and heterocyclyl. At each occurrence, each of R^{A} and R^{B} is, independently, selected from hydrogen, alkyl, alkenyl, alkynyl, cycloalkyl, cycloalkenyl, cycloalkynyl, aryl, heteroaryl, and heterocyclyl.

Each of R^{A}, R^{B} and R^{C} optionally bears one or more substituents selected from amino, alkylamino, dialkylamino, aminocarbonyl, halogen, alkyl, aryl, heteroalkyl, heteroaryl, carbocycle, heterocycle, alkylaminocarbonyl, dialkylaminocarbonyl, alkylaminocarbonyloxy, dialkylaminocarbonyloxy, nitro, cyano, carboxy, alkoxycarbonyl, alkylcarbonyl, alkoxy, haloalkoxy groups, hydroxy, protected hydroxyl groups (e.g., —O—X, where X is acyl, phenyl, substituted phenyl, benzyl, substituted benzyl, phenethyl, or substituted phenethyl), -M-heteroaryl, -M-heterocycle, -M-aryl, -M-OR^{B}, -M-SR^{B}, -M-NR^{A}R^{B}, -M—OC(O)NR^{A}R^{B}, -M-C(═NR^{B})NR^{A}R^{B}, -M-C(═NR^{A})OR^{B}, -M-P(═O)(R^{C})₂, Si(R^{C})₃, -M-NR^{A}C(O)R^{B}, -M-NR^{A}C(O)OR^{B}, -M-C(O)R^{B}, -M-C(═S)R^{B}, -M-C(═S)NR^{A}R^{B}, -M-C(O)NR^{A}R^{B}, -M-C(O)NR^{B}-M-NR^{A}R^{B}, -M-NR^{B}C(NR^{A})NR^{A}R^{B}, -M-NR^{A}C(S)NR^{A}R^{B}, -M-S(O)₂R^{A}, -M-C(O)R^{A}, -M—OC(O)R^{A}, -MC(O)SR^{B}, -M-S(O)₂NR^{A}R^{B}, —C(O)-M-C(O)R^{B}, -MCO₂R^{B}, -MC(═O)NR^{A}R^{B}, -M-C(═NH)NR^{A}R^{B}, and -M—OC(═NH)NR^{A}R^{B}, wherein M is a C₁₋₆ alkyl group. Non-limiting illustrations of a substituted R^{A}, R^{B} or R^{C} group include haloalkyl and trihaloalkyl, alkoxyalkyl, halophenyl, chloromethyl, trichloromethyl, trifluoromethyl, methoxyethyl, alkoxyphenyl, halophenyl, —CH₂-aryl, —CH₂-heterocycle, —CH₂C(O)NH₂, —C(O)CH₂N(CH₃)₂, —CH₂CH₂OH, —CH₂OC(O)NH₂, —CH₂CH₂NH₂, —CH₂CH₂CH₂NEt₂, —CH₂OCH₃, —C(O)NH₂, —CH₂CH₂-heterocycle, —C(═S)CH₃, —C(═S)NH₂, —C(═NH)NH₂, —C(═NH)OEt, —C(O)NH-cyclopropyl, —C(O)NHCH₂CH₂-heterocycle, —C(O)NHCH₂CH₂OCH₃, —C(O)CH₂CH₂NHCH₃, —CH₂CH₂F, —C(O)CH₂-heterocycle, —CH₂C(O)NHCH₃, —CH₂CH₂P(═O)(CH₃)₂, and —Si(CH₃)₃.

An alkyl, alkenyl, alkynyl, alkoxy, haloalkyl, heteroalkyl, cycloalkyl, cycloalkenyl, cycloalkynyl, or heterocyclic group may contain one or more substituents selected from those listed above for aryl and heteroaryl groups, in addition to ═O, αS, ═NH, ═NNR^{A}R^{B}, ═NNHC(O)R^{B}, ═NNHCO₂R^{B}, or ═NNHSO₂R^{B}, wherein R^{A} and R^{B} are as defined above. Non-limiting examples of substituents on a nitrogen, e.g., within a heterocyclic or other moiety, include alkyl (substituted or unsubstituted), acyl, aminoacyl and sulfonyl groups.

Other features and advantages of the invention will be apparent from the following detailed description and the claims.

### DETAILED DESCRIPTION

The invention features a method for treating patients who have an EGFR-driven cancer, which is, or has become, refractory to erlotinib or gefitinib, or which bears an EGFR mutation identified herein, by administering a compound of formula (I) to the patient.

### EGFR Mutants

EGFR mutants include one or more deletions, substitutions, or additions in the amino acid or nucleotide sequences of EGFR, or fragments thereof.

Mutations in EGFR can occur in any part of the EGFR sequence. Generally, EGFR mutants arise from mutations in the kinase domain (i.e., exons 18-24 in the EGFR sequence) or in the extracellular domain (i.e., exons 2-16 in the EGFR sequence). For example, mutations typically occur in the kinase domain, including one or more of a point mutation in exon 18 (e.g., L688P, V689M, P694L/S, N700D, L703V, E709K/Q/A/GN, 1715S, L718P, G719C/A/S/R, or S720P/F), a deletion in exon 19 that may or may not include an insertion (e.g., delG719, delE746_E749, delE746_A750, delE746_A750insRP, delE746_A750insQP, delE746_T751, delE746_T751insA/I/V, delE746_T751insVA, delE746_S752, delE746_S752insA/V/D, delE746_P53insLS, delL747_E749, delL747_A750, delL747_A750insP, delL747_T751, delL747_T751insP/S/Q, delL747_T751insPI, delL747_S752, delL747_S752insQ, delL747_P753, delL747_P753insS/Q, delL747_L754insSR, delE749_A750, delE749_A750insRP, delE749_T751, delT751_I759, delT751_I759insS/N, or delS752_I759), a duplication in exon 19 (e.g., K739_I44dupKIPVAI), a point mutation in exon 19 (e.g., L730F, W731Stop, P733L, G735S, V742A, E746V/K, A750P, T751I, S752Y, P753S, A754P, or D761Y), an in-frame insertion in exon 20 (e.g., D761_E762insEAFQ, A767_S768insTLA, V769_D770insY, V769_D770insCV, V769_D770insASV, D770_N771insD/G, D770_N771insNPG, D770_N771insSVQ, P772_H773insN/V, P772_H773insYNP, or V774_C775insHV), a deletion in exon 20 that may or may not include an insertion (e.g., delM766_A767, delM766_A767insAI, delA767_V769, delD770, or delP772_H773insNP), a duplication in exon 20 (e.g., S768_D770dupSVD, A767_V769dupASV, or H773dupH), a point mutation in exon 20 (e.g., D761N, A763V, V765A/M, S768I, V769L/M, S768I, P772R, N771T, H773R/V/L, V774M, R776G/H/C, G779S/F, T783A, T784F, L792P, L798H/F, T790M, R803W, K806E, or L814P), or a point mutation in exon 21 (e.g., G810S, N826S, L833V, H835L, L838V, A839T, K846R, T847I, H850N, V851I/A, 1853T, L858M/R, A859T, L861Q/R, G863D, A864T, E866K, or G873E). In lung cancer, activation mutants are typical, and 90% deletion of 746-750 (ELREA) and L858R result in sustained phosphorylation of EGFR without ligand stimulation. Drug resistance in 50% of lung cancers is said to arise from the T790M point mutation.

For example, in glioblastoma, mutations typically, but not exclusively, occur in the extracellular domain, including EGFR variant I (EGFRvI) lacking the extracellular domain and resembling the v-erbB oncoprotein; EGFRvII lacking 83 amino acids from domain IV; and EGFRvIII lacking amino acids 30-297 from domains I and II, which is the most common amplification and is reported in 30-50% of glioblastomas and 5% of squamous cell carcinoma. Other mutations for glioblastoma include one or more of point mutations in exon 2 (e.g., D46N or G63R), exon 3 (e.g., R108K in domain I), exon 7 (e.g., T263P or A289D/T/V in domain II), exon 8 (e.g., R324L or E330K), exon 15 (e.g., P596L or G598V in domain IV), or exon 21 (L861Q in the kinase domain).

EGFR mutants also include those with a combination of two or more mutations, as described herein. Exemplary combinations include S768I and G719A; S768I and V769L; H773R and W731Stop; R776G and L858R; R776H and L861Q; T790M and L858R; T790M and delE746_A750; R803W and delE746_{—}751insVA; delL747_E749 and A750P; delL747_S752 and E746V; delL747_S752 and P753S; P772_H773insYNP and H773Y; P772_H773insNP and H773Y; and D770_N771insG and N771T. Combinations of particular current interest include combinations of T790M together with another mutation (e.g., T790M and L858R or T790M and delE746_A750).

Certain mutations encode mutant EGFR proteins that actively signal in the absence of an EGF ligand but which are characterized by sensitivity to EGFR inhibitors such as gefitinib and erlotinib. G719C/S/A, delE746_A750, and L858R are examples of such mutations. Other EGFR mutations confer resistance to such drugs, even when present in combination with one of the previously mentioned activating mutations. T790M is an example of a mutation that confers resistance to those drugs.

EGFR-driven cancers may be driven by a wild-type EGFR or by any mutant EGFRs described herein. For example, EGFRvIII is commonly found in glioblastoma and has also been reported in breast, ovarian, prostate, and lung carcinomas. Exemplary EGFR-driven cancers: glioblastoma, lung cancer (e.g., squamous cell carcinoma, non-small cell lung cancer, adenocarcinoma, bronchioloalveolar carcinoma (BAC), BAC with focal invasion, adenocarcinoma with BAC features, and large cell carcinoma), pancreatic cancer, head and neck cancers (e.g., squamous cell carcinoma), breast cancer, colorectal cancer, epithelial cancer (e.g., squamous cell carcinoma), ovarian cancer, and prostate cancer.

In particular, the invention described herein will be of interest for patients who have, or have a higher risk of, a TKI-resistant EGFR mutation. About 8,000 to 16,000 new cases per year can be estimated based on: incidence of non-small cell lung cancer (about 160,000 new cases in the U.S.), the response to erlonitinib in the general population (about 10%, resulting in a sensitive population of 16,000), the presence of activation mutations (10-20% in white and 30-40% in Asian population, resulting in a sensitive population of 16,000-32,000), acquisition of secondary resistance (most if not all patients, resulting in a sensitive population of 16,000-32,000), and percentage of patients carrying the T790M point mutations (about 50%, resulting in a sensitive population of 8,000-16,000). Patients having TKI-resistant mutations include those patients having cancers resistant to one or more of erlotinib, gefitinib, CL-387,785, BIBW 2992 (CAS Reg. No. 439081-18-2), CI-1033, neratinib (HKI-272), MP-412 (AV-412), PF-299804, AEE78, and XL64.

In particular, the inventions relates to treatment of EGFR-driven cancers having the T790M point mutation. Generally, reversible inhibitors (e.g., CI-1033, neratinib (HKI-272), and PF-299804) are less potent in cell lines having the T790M mutation and do not inhibit T790M at clinically achievable concentrations. Since the ATP Km of T790M and WT are similar, concentrations that inhibit the mutant will inhibit the WT and result in gastrointestinal and cutaneous events.

An EGFR mutant also includes other amino acid and nucleotide sequences of EGFR with one or more deletions, substitutions, or additions, such as point mutations, that retain or increase tyrosine kinase or phosphorylation activity. Where the mutant is a protein or polypeptide, preferable substitutions are conservative substitutions, which are substitutions between amino acids similar in properties such as structural, electric, polar, or hydrophobic properties. For example, the substitution can be conducted between basic amino acids (e.g., Lys, Arg, and His), or between acidic amino acids (e.g., Asp and Glu), or between amino acids having non-charged polar side chains (e.g., Gly, Asn, Gln, Ser, Thr, Tyr, and Cys), or between amino acids having hydrophobic side chains (e.g., Ala, Val, Leu, Ile, Pro, Phe, and Met), or between amino acids having branched side chains (e.g., Thr, Val, Leu, and Ile), or between amino acids having aromatic side chains (e.g., Tyr, Trp, Phe, and His).

Where the mutant is a nucleic acid, the DNA encoding an EGFR mutant protein may comprise a nucleotide sequence capable of hybridizing to a complement sequence of the nucleotide sequence encoding an EGFR mutant, as defined herein, under stringent conditions. As used herein, the stringent conditions include low, medium or high stringent conditions. An example of the stringent conditions includes hybridization at approximately 42-55° C. in approximately 2-6×SSC, followed by wash at approximately 50-65° C. in approximately 0.1-1×SSC containing approximately 0.1-0.2% SDS, where 1×SSC is a solution containing 0.15 M NaCl and 0.015 M Na citrate, pH 7.0. Wash can be performed once or more. In general, stringent conditions may be set at a temperature approximately 5° C. lower than a melting temperature (Tm) of a specific nucleotide sequence at defined ionic strength and pH.

The amino acid and nucleotide sequences of EGFR and DNAs encoding them are available from well known databases such as NCBI GenBank (USA), EMBL (Europe), etc. For example, GenBank accession numbers for EGFR [Homo sapiens] include MIM131550, AAI28420, NM_{—}005228, NP_{—}005219.2, and GeneID: 1956.

### Characterization of EGFR-Driven Cancers

EGFR mutant expression or overexpression can be determined in a diagnostic or prognostic assay by evaluating levels of EGFR mutants in biological sample, or secreted by the cell (e.g., via an immunohistochemistry assay using anti-EGFR antibodies or anti-p-EGFR antibodies; FACS analysis, etc.). Alternatively, or additionally, one can measure levels of EGFR mutant-encoding nucleic acid or mRNA in the cell, e.g., via fluorescent in situ hybridization using a nucleic acid based probe corresponding to an EGFR mutant-encoding nucleic acid or the complement thereof; (FISH; see WO98/45479, published October, 1998), Southern blotting, Northern blotting, or polymerase chain reaction (PCR) techniques, such as real time quantitative PCR(RT-PCR). One can also study EGFR mutant expression by measuring shed antigen in a biological sample, such as serum, e.g., using antibody-based assays (see also, e.g., U.S. Pat. No. 4,933,294, issued Jun. 12, 1990; WO91/05264, published Apr. 18, 1991; U.S. Pat. No. 5,401,638, issued Mar. 28, 1995; and Sias et al., J. Immunol. Methods 132:73 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

Examples of biological properties that can be measured in isolated cells include mRNA expression, protein expression, and DNA quantification. Additionally, the DNA of cells isolated by the methods of the invention can be sequenced, or certain sequence characteristics (e.g., polymorphisms and chromosomal abnormalities) can be identified using standard techniques, e.g., FISH or PCR. The chemical components of cells, and other analytes, may also be assayed after isolation. Cells may also be assayed without lysis, e.g., using extracellular or intracellular stains or by other observation, e.g., morphology or growth characteristics in various media.

While any hybridization technique can be used to detect the gene rearrangements, one preferred technique is fluorescent in situ hybridization (FISH). FISH is a cytogenetic technique which can be used to detect and localize the presence or absence of specific DNA or RNA sequences on chromosomes. FISH incorporates the use of fluorescently labeled nucleic acid probes which bind only to those parts of the chromosome with which they show a high degree of sequence similarity. Fluorescence microscopy can be used to find out where the fluorescent probe bound to the chromosome. The basic steps of FISH are outlined below. Exemplary FISH probes include Vysis EGFR SpectrumOrange/CEP SpectrumGreen Probe (Abbott, Downers Grove, Ill.), which hybridizes to band 7p12; and ZytoLight SPEC EGFR/CEN 7 Dual Color Probe (ZytoVision), which hybridizes to the alpha-satellite sequences of the centromere of chromosome 7.

For FISH, a probe is constructed that is long enough to hybridize specifically to its target (and not to similar sequences in the genome), but not too large to impede the hybridization process. Probes are generally labeled with fluorophores, with targets for antibodies, with biotin, or any combination thereof. This can be done in various ways, for example using random priming, nick translation, and PCR using tagged nucleotides.

Generally, a sample or aliquot of a population of cells is used for FISH analysis. For example, in one method of preparation, cells are trypsinized to disperse into single cells, cytospun onto glass slides, and then fixed with paraformaldehyde before storing in 70% ethanol. For preparation of the chromosomes for FISH, the chromosomes are firmly attached to a substrate, usually glass. After preparation, the probe is applied to the chromosome RNA and starts to hybridize. In several wash steps, all unhybridized or partially hybridized probes are washed away. If signal amplification is necessary to exceed the detection threshold of the microscope (which depends on many factors such as probe labeling efficiency, the kind of probe, and the fluorescent dye), fluorescent tagged antibodies or strepavidin are bound to the tag molecules, thus amplifying the fluorescence.

An epifluorescence microscope can be used for observation of the hybridized sequences. The white light of the source lamp is filtered so that only the relevant wavelengths for excitation of the fluorescent molecules arrive onto the sample. Emission of the fluorochromes happens, in general, at larger wavelengths, which allows one to distinguish between excitation and emission light by mean of another optical filter. With a more sophisticated filter set, it is possible to distinguish between several excitation and emission bands, and thus between several fluorochromes, which allows observation of many different probes on the same strand.

Depending on the probes used, FISH can have resolution ranging from huge chromosomes or tiny (˜100 kilobase) sequences. The probes can be quantified simply by counting dots or comparing color.

Allele-specific quantitative real time-PCR may also be used to identify a nucleic acid encoding a mutant EGFR protein (see, for e.g., Diagnostic Innovations DxS BCR-ABL T3151 Mutation Test Kit, and Singer et al., Methods in Molec. Biol. 181:145 (2001)). This technique utilizes Taq DNA polymerase, which is extremely effective at distinguishing between a match and a mismatch at the 3′-end of the primer (when the 3′-base is mismatched, no efficient amplification occurs). Using this technique, the 3′-end of the primer may be designed to specifically hybridize to a nucleic acid sequence that corresponds to a codon that encodes a mutant amino acid in an EGFR mutant, as described herein. In this way, the specific mutated sequences can be selectively amplified in a patient sample. This technique further utilizes a Scorpion probe molecule, which is a bifunctional molecule containing a PCR primer, a fluorophore, and a quencher. The fluorophore in the probe interacts with a quencher, which reduces fluorescence. During a PCR reaction, when the Scorpion probe binds to the amplicon, the fluorophore and quencher in the Scorpion probe become separated, which leads to an increase in fluorescence from the reaction tube. Any of the primers described herein may be used in allele-specific quantitative real time PCR.

A biological sample can be analyzed to detect a mutation in an EGFR gene, or expression levels of an EGFR gene, by methods that are known in the art. For example, methods such as direct nucleic acid sequencing, altered hybridization, aberrant electrophoretic gel migration, binding or cleavage mediated by mismatch binding proteins, single-strand conformational polymorphism (SSCP) analysis, or restriction fragment length polymorphism (RFLP) analysis of PCR products derived from a patient sample can be used to detect a mutation in an EGFR gene; ELISA can be used to measure levels of EGFR polypeptide; and PCR can be used to measure the level of an EGFR nucleic acid molecule.

Any of these techniques may be used to facilitate detection of a mutation in a candidate gene, and each is well known in the art; examples of particular techniques are described, without limitation, in Orita et al. (Proc. Natl. Acad. Sci. USA 86:2766 (1989)) and Sheffield et al. (Proc. Natl. Acad. Sci. USA 86:232 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, N.Y. (1995); PCR Technology: Principles and Applications for DNA Amplification, H. A. Ehrlich, Ed., Stockton Press, NY; Yap et al., Nucl. Acids. Res. 19:4294 (1991)).

One skilled in the art may identify in a nucleic acid or protein sequence a residue (e.g., amino acid or nucleotide) or codon that corresponds to a residue or codon in wild-type EGFR or EGFR mutants using a number of sequence alignment software programs (e.g., NCBI BLAST website). Such software programs may allow for gaps in the alignment of the compared sequences. Using such software, one skilled in the art may identify a nucleotide, amino acid, or amino acid that corresponding to a specific nucleotide, amino acid, or codon in wild-type EGFR or EGFR mutants.

Levels of EGFR expression (e.g., DNA, mRNA, or protein) in a biological sample can be determined by using any of a number of standard techniques that are well known in the art or described herein. Exemplary biological samples include plasma, blood, sputum, pleural effusion, bronchoalveolar lavage, or biopsy, such as a lung biopsy and lymph node biopsy. For example, EGFR expression in a biological sample (e.g., a blood or tissue sample) from a patient can be monitored by standard northern blot analysis or by quantitative PCR (see, e.g., Ausubel et al., supra; PCR Technology: Principles and Applications for DNA Amplification, H. A. Ehrlich, Ed., Stockton Press, NY; Yap et al., Nucl. Acids. Res. 19:4294 (1991)).

### Synthesis of Compounds

Compounds of formula (I) can be prepared using known methods and materials, e.g., as disclosed in detail in International patent applications WO 2004/080980, WO 2005/016894, WO 2006/021454, WO 2006/021457, WO 2009/143389, and WO 2009/126515. For instance, compounds of formula (I) in which R^{e} is H and R^{d} is H, Cl, CF₃, or CH₃, can be synthesized from 2,4-dichloropyrimidine, 2,4,5-trichloropyrimidine,
2,4-dichloro-5-(trifluoromethyl)pyrimidine, or 2,4-dichloro-5-methylpyrimidine, respectively, as described in PCT Publication No. WO/2009/143389 (see, for example, Schemes 1A and 1B below).

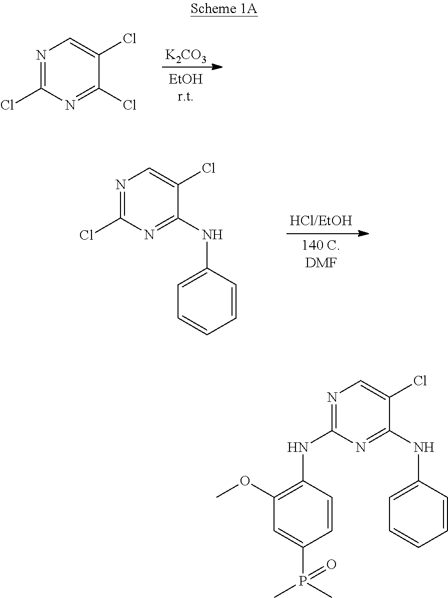

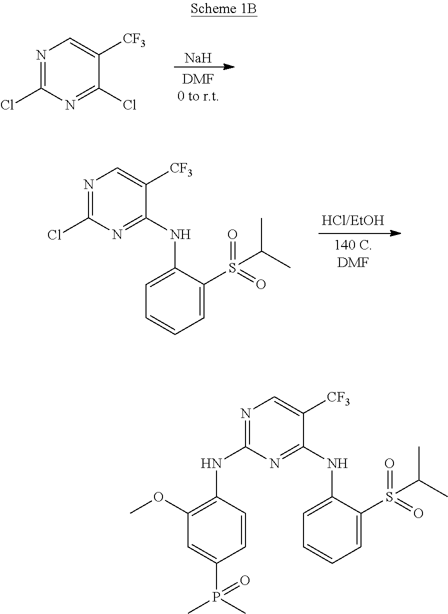

Compounds of formula (I) in which R^{d} and R^{e}, together with the pyrimidine ring atoms to which they are attached, form a 5- or 6-membered ring containing one or two heteroatoms can be synthesized as described in PCT Publication No. WO2009/126515. See, for example, Scheme 2, which describes the synthesis of a starting material from which compounds of formula (I) can be synthesized. In Scheme 2, X is CH₃ or H.

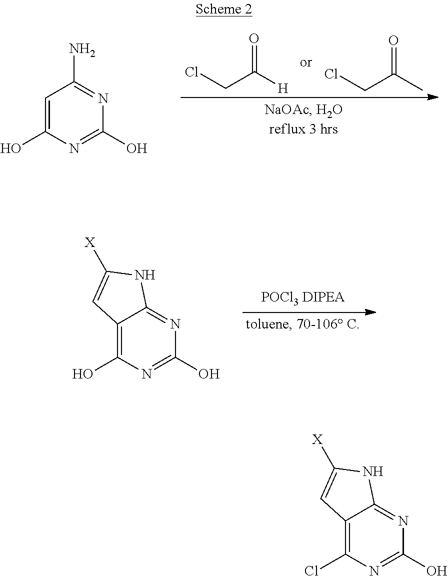

### Formulation

Compounds of formula (I) can be formulated for any route of administration (e.g., orally, rectally, parenterally, intracisternally, intravaginally, intraperitoneally, topically (as by transdermal patch, powders, ointments, or drops), sublingually, bucally, as an oral or nasal spray) effective for use in the methods of the invention. For use in the methods of the invention, compounds of formula (I) are preferably formulated in dosage unit form for ease of administration and uniformity of dosage. For example, a compound of formula (I) can be formulated for as a capsule for oral administration containing nominally 10 mg, 50 mg, 100 mg, 150 mg, 250 mg, 500 mg, or any dosage amounts described herein as the free base or acid addition salt of the compound (e.g., the hydrochloride salt). The unit dosage forms of the invention can include the compound, or a salt thereof, formulated with fillers, flow enhancers, lubricants, and/or disintegrants as needed. For example, a unit dosage form can include colloidal silicon dioxide (a flow enhancer), lactose anhydrous (a filler), magnesium stearate (a lubricant), microcrystalline cellulose (a filler), and/or sodium starch glycolate (a disintegrant). The compound and the inactive ingredients can be formulated utilizing, for example, conventional blending, and encapsulation processes. Alternatively, compounds of formula (I) are formulated as described in PCT Publication Nos. WO2009/143389 and WO2009/126515.

### Therapy

Compounds of formula (I) can be useful for treating EGFR-driven cancers. In particular, the compounds can be useful for treating EGFR-driven cancers that express EGFR mutants and for treating EGFR-driven cancers that are refractory to TKI therapies (e.g., erlotinib or gefitinib).

Such cancers can include, among others, non-small cell lung cancer (NSCLS), including one or more of squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; adenocarcinomas; and including cancers which are EGFR mediated.

The present invention is based upon the discovery that compounds of formula (I) can be used to treat EGFR-driven cancers, EGFR-driven cancers that express EGFR mutants, and for treating EGFR-driven cancers that are refractory to TKI therapy, such as erlotinib or gefitinib. Compounds of formula (I) can also be used in a maintenance role to prevent recurrence of cancer in patients in need of such a treatment.

The therapeutically effective dose of a compound of formula (I) will often be in the range of an average daily dose of from 5 mg to 2,000 mg of compound administered in single or multiple doses to an adult patient, preferably orally. Typical average daily dose ranges include 10-500 mg, 20-550 mg, 30-600 mg, 40-650 mg, 50-700 mg.

Administration may be once or multiple times daily, weekly (or at some other multiple-day interval) or on an intermittent schedule. For example, the compound may be administered one or more times per day on a weekly basis (e.g. every Monday) indefinitely or for a period of weeks, e.g. 4-10 weeks. Alternatively, it may be administered daily for a period of days (e.g. 2-10 days) followed by a period of days (e.g. 1-30 days) without administration of the compound, with that cycle repeated indefinitely or for a given number of repetitions, e.g. 4-10 cycles. As an example, a compound of the invention may be administered daily for 5 days, then discontinued for 2 days, then administered daily for another 5 day period, then discontinued for 2 days, and so on, repeating the cycle indefinitely, or for a total of 4-10 times.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

Reagents: The following compounds were synthesized or purchased for screening: WZ4003 (Zhou et al., Nature, 462:1070 (2009)), HKI-272, and CI-387,785.

### Example 1

### Kinase Assays

Assays were conducted for an in vitro kinase panel having WT EGFR, L858R, T790M, and L858R/T790M. Additional assays can be conducted with the panel further including delE746_A750 and delE746_A750/T790M. Assay conditions included 10 pt curves with 3 μM top concentration (singlicates) and 10 μM ATP.

Compounds formula (I) included potent inhibitors of EGFR mutants in kinase assays. For example, in the H1975 cell line having L858R and T790M mutations, previously known inhibitors gefitinib, CL-387,785, and HKI-272 had IC50 values between 153 nM to >3.3 μM, while many compounds of formula (I) exhibited IC50 values in the range of 0.5 to 9 nM. Thus, compounds of formula (I) could provide the necessary inhibitors for EGFR-driven cancers.

### Example 2

### Cellular and In Vivo Assays

NSCLC cell lines as well as engineered Ba/F3 and NIH3T3 cell lines were used to examine the activity of compounds of formula (I) against 3 general forms of EGFR: native EGFR (the naturally occurring form), EGFR with an activating mutation (delE746_A750 [Del] or L858R; this form is sensitive to first generation EGFR inhibitors), and EGFR with both an activating mutation and a T790M resistance mutation (L858R/T790M or Del/T790M; the addition of the T790M mutation makes this form resistant to first generation EGFR inhibitors). Effects of test compounds on EGFR signaling were assessed by measuring levels of phosphorylated EGFR, effects on in vitro proliferation measured by a growth or viability assay, and effects on in vivo tumor growth measured in mice following daily oral dosing.

Test compounds of greatest interest were essentially inactive against native EGFR in cellular assays, i.e., inhibited phosphorylation with IC50s>1000 nM in a NSCLC cell line lacking an activating mutation in EGFR and in native EGFR-transduced NIH3T3 cells.

In contrast, test compounds demonstrated potent activity against activated forms of EGFR in both in vitro and in vivo cellular assays. EGFR phosphorylation was inhibited with IC50s of in some cases under ˜65 nM across 3 cell lines: a NSCLC line expressing EGFR-Del, and NIH3T3 cells expressing EGFR-Del or EGFR-L858R. In NSCLC cells [Del], cell growth was inhibited with a G150 of under ˜200 nM. Xenograft experiments with that NSCLC cell line [Del] showed in some case that doses of 25 mg/kg or greater induced tumor regression by >33% and inhibited EGFR signaling by >85% and >40% at 10 and 24 hours after dosing, respectively.

Test compounds of interest also demonstrated potent activity against T790M mutant forms of EGFR in in vitro cellular assays. In one set of studies, EGFR signaling was inhibited with IC50s of below ˜65 nM across 6 cell lines: NSCLC lines expressing EGFR-L858R/T790M (H1975) or EGFR-Del/T790M (engineered HCC827 cells), and pairs of NIH3T3 cells and Ba/F3 cells expressing either EGFR-Del/T790M or EGFR-L858R/T790M. Viability of the two Ba/F3 cell lines was inhibited with IC50s of 141 and 502 nM. Growth of HCC827 cells [Del] engineered to express EGFR-Del/T790M was inhibited with a G150 (245 nM) similar to that of the parental HCC827 cells that express EGFR-Del. In contrast, the potency of erlotinib was reduced by >100-fold in cells expressing EGFR-Del/T790M versus cells expressing EGFR-Del.

Lastly, an exemplary test compound also exhibited potent activity against T790M mutant forms of EGFR in in vivo assays. In a tumor model using Ba/F3 cells expressing EGFR-Del/T790M, daily oral dosing with 50 mg/kg AP26113 inhibited growth by >90% and dosing with 75 mg/kg induced tumor regression. A single dose of 50 mg/kg was shown to inhibit levels of phosphorylated EGFR in the tumor by >80% 24 h after dosing. Antitumor and anti-EGFR activity was also seen in a tumor model using NIH3T3 cells expressing EGFR-Del/T790M.

### Example 3

### Cellular Inhibition Assays

Lung cancer cell lines were analyzed by determining phosphorylation and expression levels of various proteins. An immunoblot analysis of phosphorylation and expression levels of EGFR and other proteins in lung cancer cells was performed for lung cancer cell lines having different EGFR mutants. H358 expresses WT EGFR, HCC827 has a delE746_A750 mutation, H820 has delE746_E749/T790M mutations, and H1975 has L858R/T790M mutations.

Immunoblot analyses were conduct for various compounds, including erlotinib, gefitinib, BIBW 2992, WZ4003, and several compounds of formula (I).

The immunoblot show that test compounds of formula (I) are potent inhibition of cancer cell lines having EGFR mutations. In particular, these compounds were efficacious against mutations generally associated with drug resistance, such as T790M and the combination of L858R and T790M.

### Example 4

### Exemplary Compounds of Formula (I)

The compounds depicted below were tested by in vitro kinase assay to determine relative inhibitory activities against native EGFR, EGFR bearing the activating L858R mutation, EGFR bearing the (resistance conferring) T790M mutation, and EGFR bearing the L858R and T790M mutations. The observed IC50 values were as follows:

| | IC50 (nanomolar) |
|---|---|
| EGFR | single digit to >1000 |
| L858R | 0.5 to 200-300 |
| T790M | single digit to >1000 |
| L858R + T790M | single digit to >1000 |

In some cases, the compounds exhibited 100-fold greater potency against the L858R mutant relative to native EGFR, and 10-fold greater potency against the double mutant relative to native EGFR.

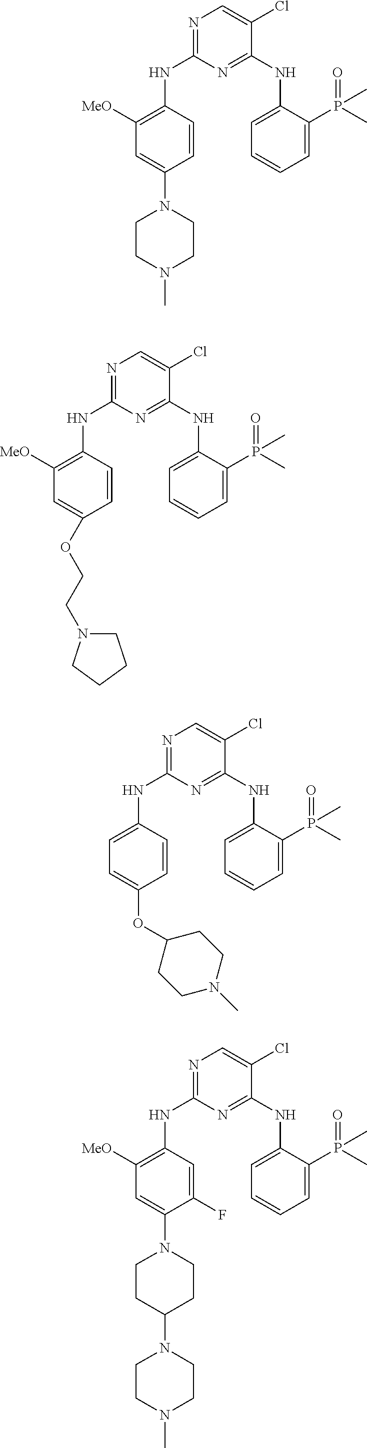

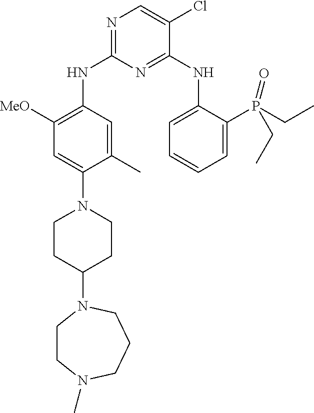 [SMILES: CCP(=O)(CC)c1ccccc1Nc1nc(Nc2cc(C)c(N3CCC(N4CCCN(C)CC4)CC3)cc2OC)ncc1Cl]

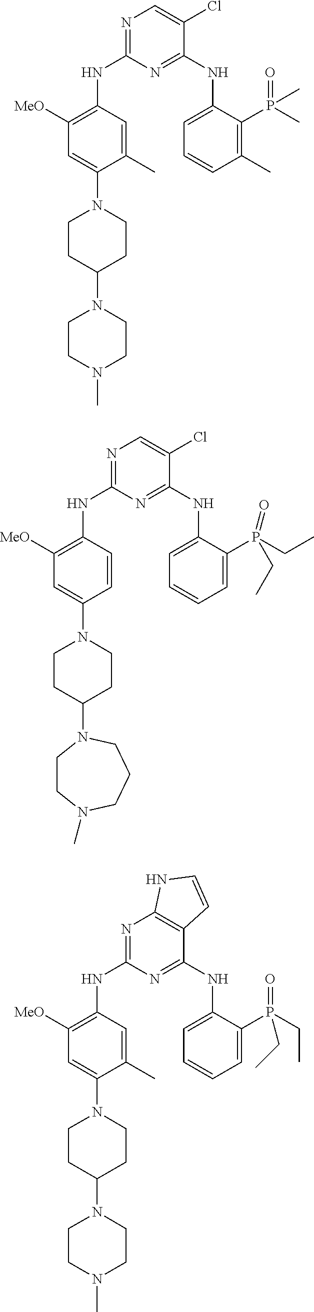 [SMILES: COc1cc(N2CCC(N3CCN(C)CC3)CC2)c(C)cc1Nc1ncc(Cl)c(Nc2cccc(C)c2P(C)(C)=O)n1 ; CCP(=O)(CC)c1ccccc1Nc1nc(Nc2ccc(N3CCC(N4CCCN(C)CC4)CC3)cc2OC)ncc1Cl ; CCP(=O)(CC)c1ccccc1Nc1nc(Nc2cc(C)c(N3CCC(N4CCN(C)CC4)CC3)cc2OC)nc2[nH]ccc12]

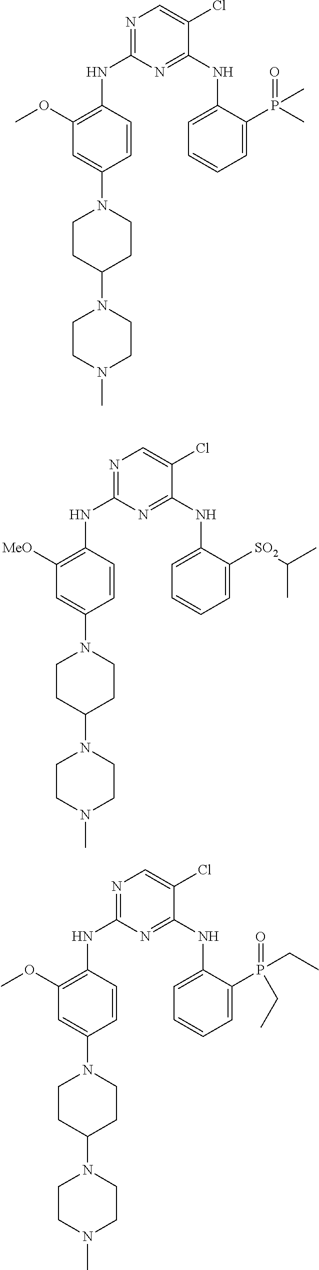

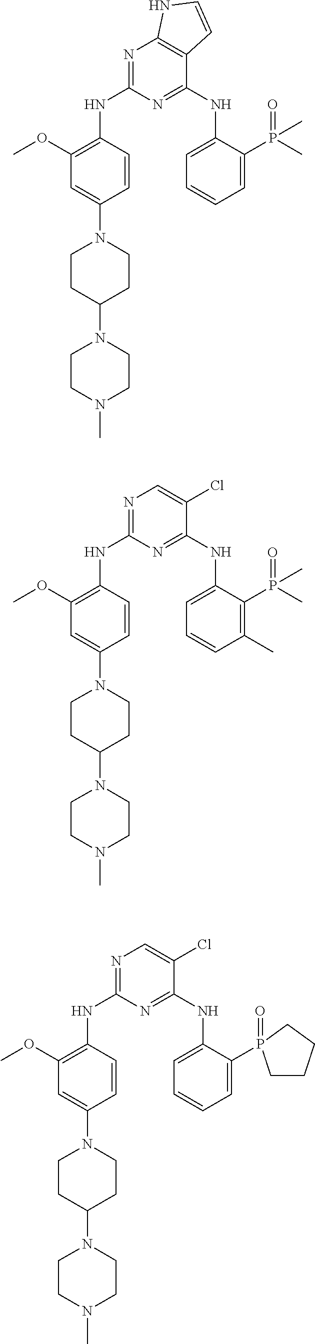

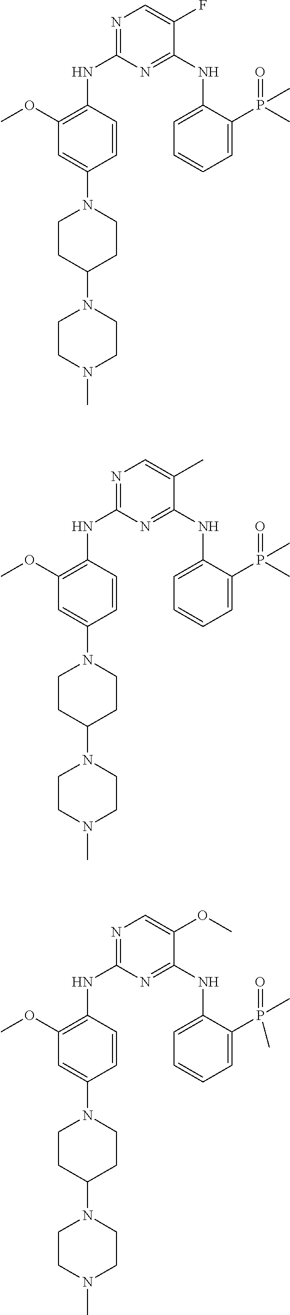

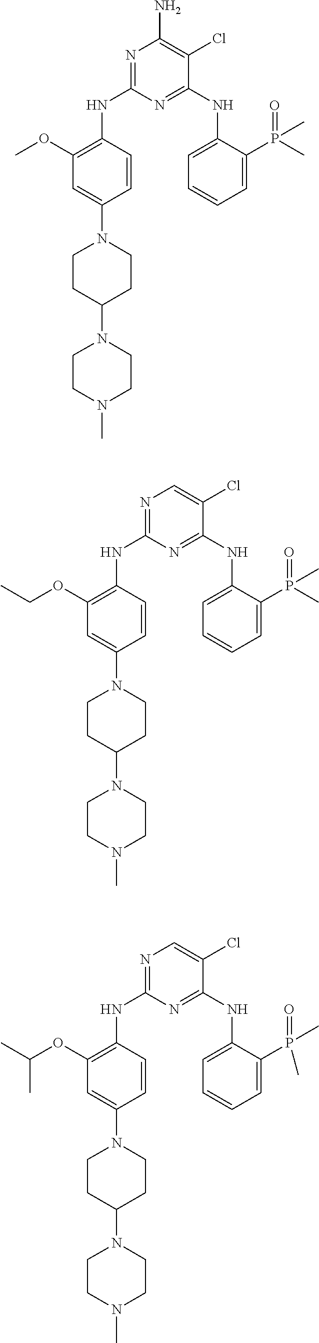

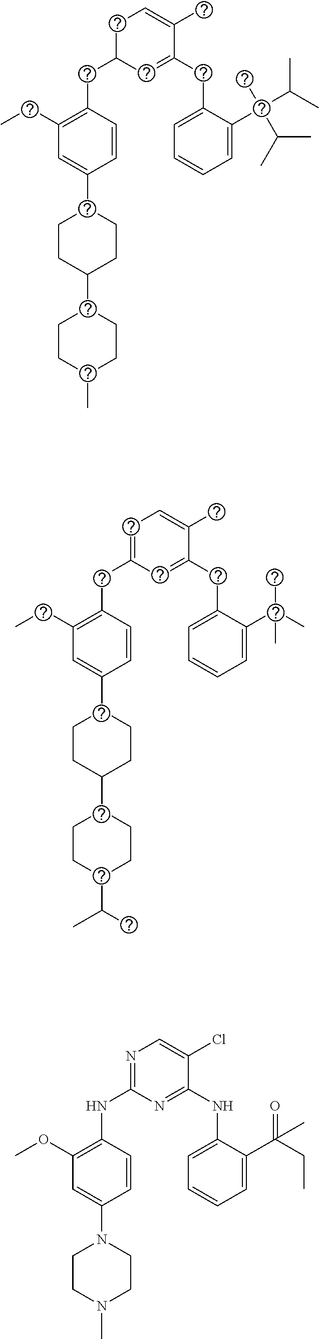

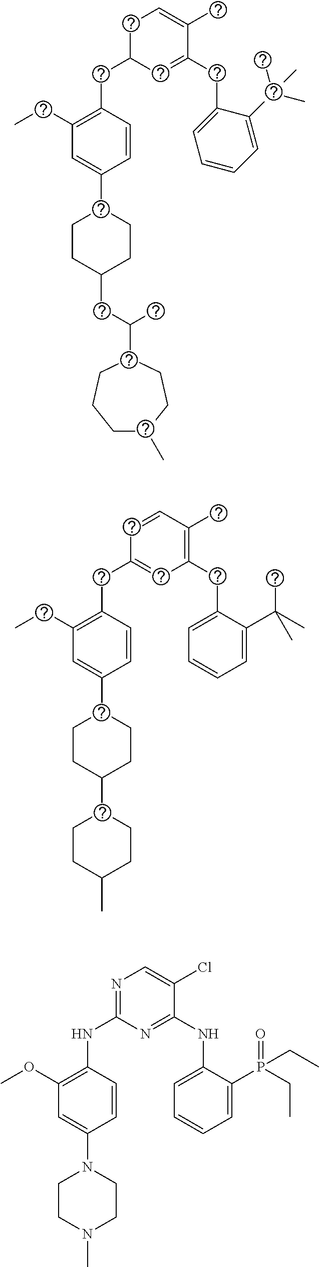

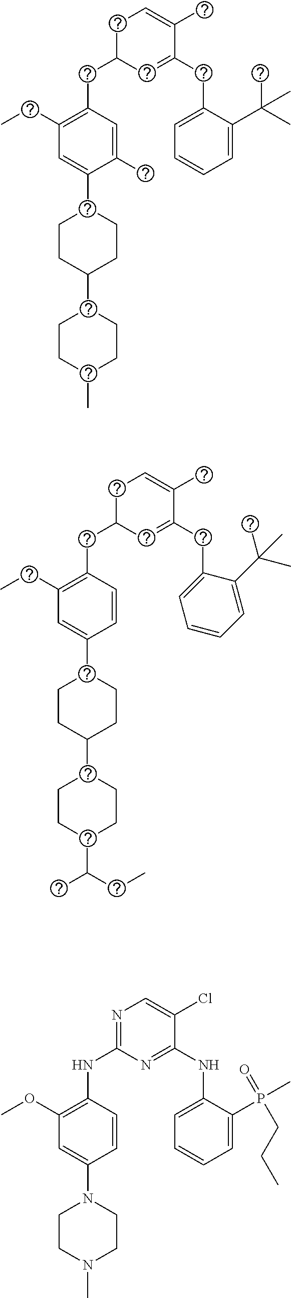

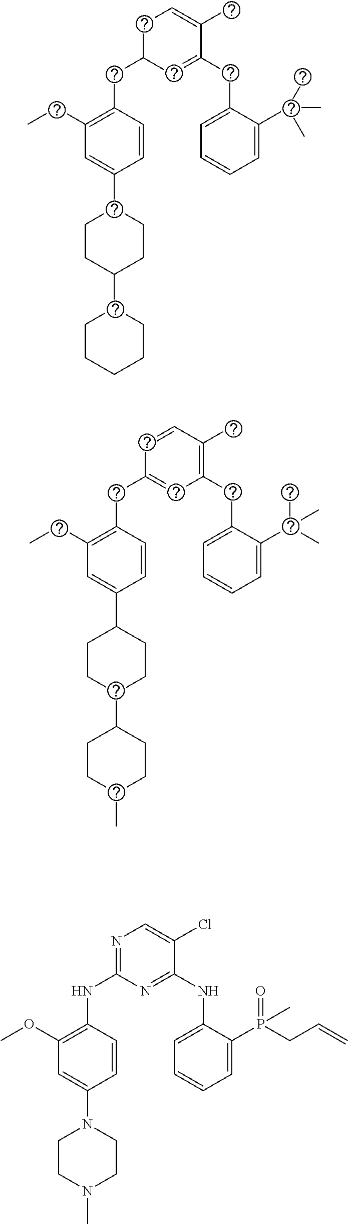

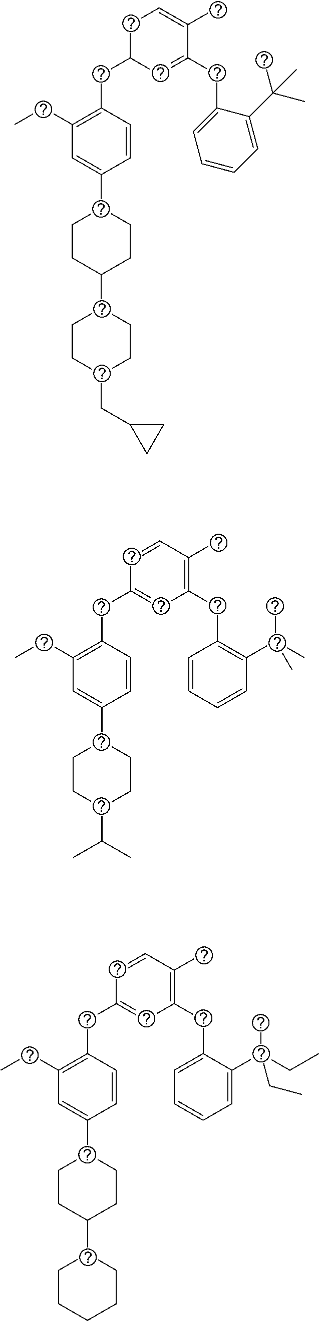

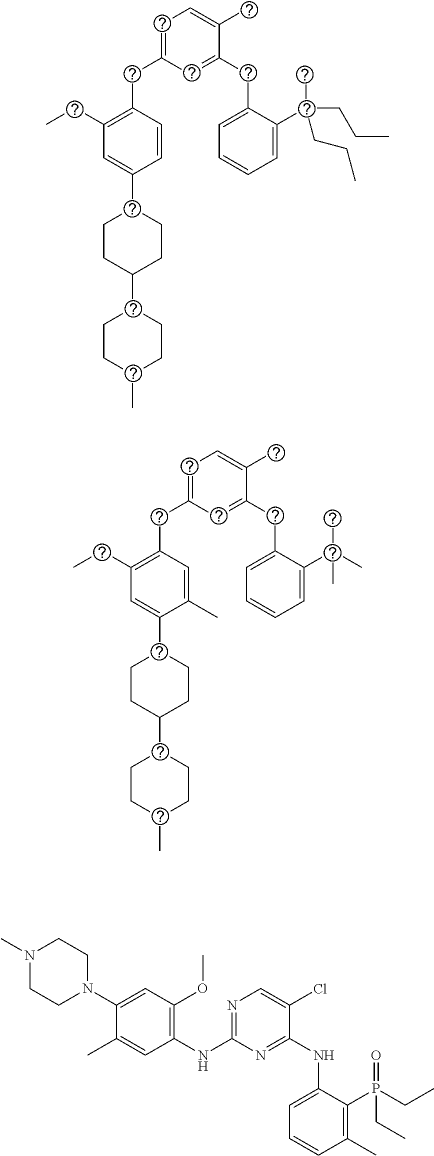

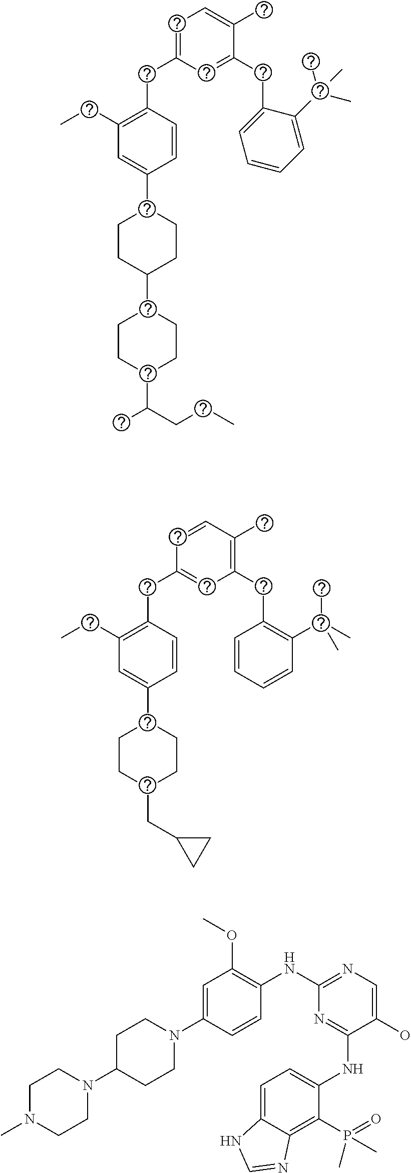

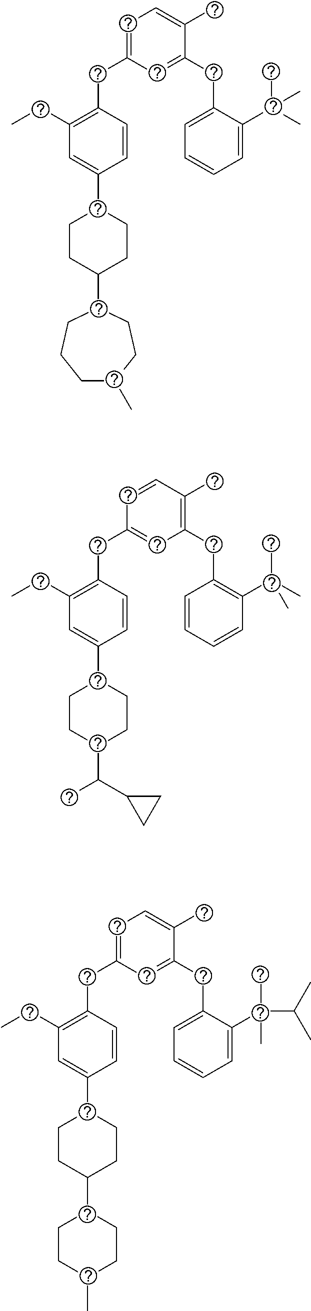

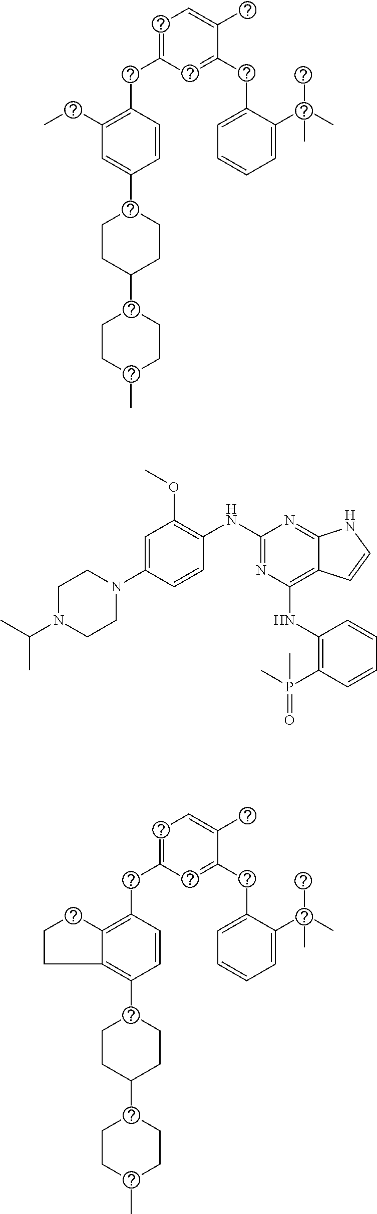

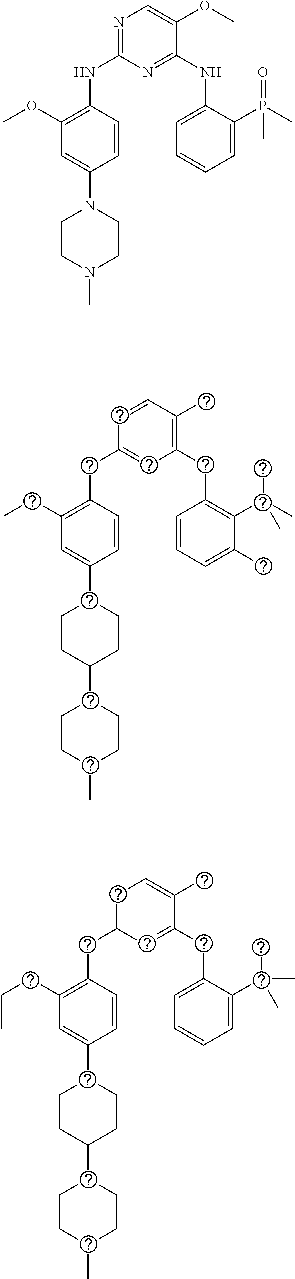

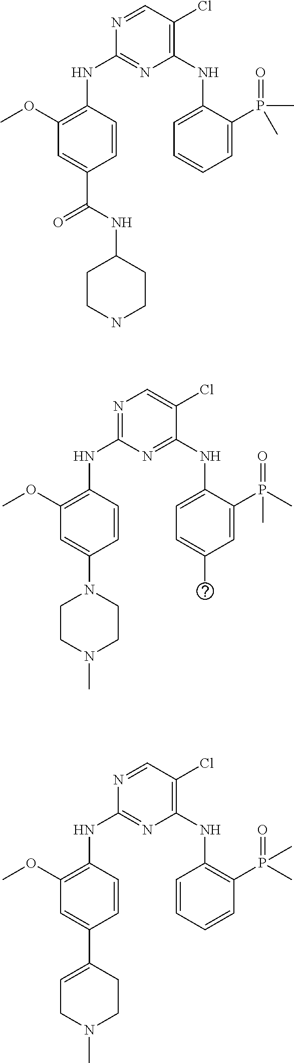

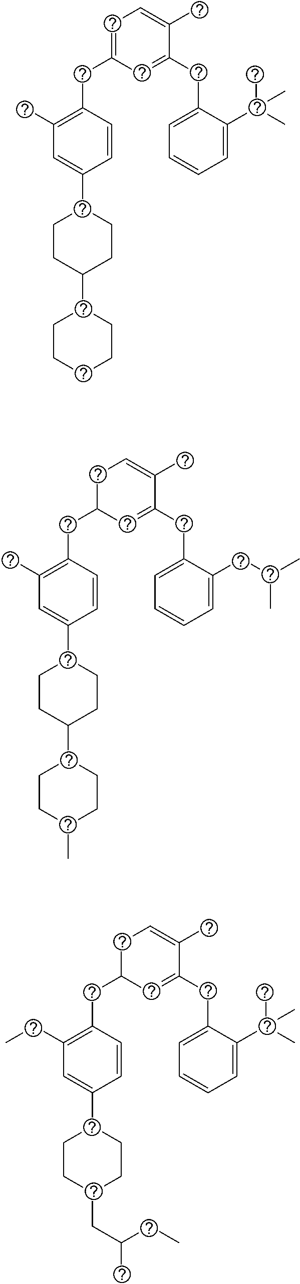

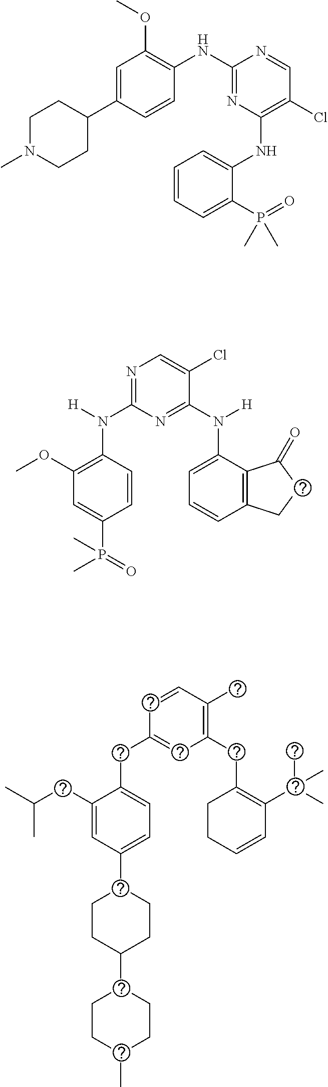

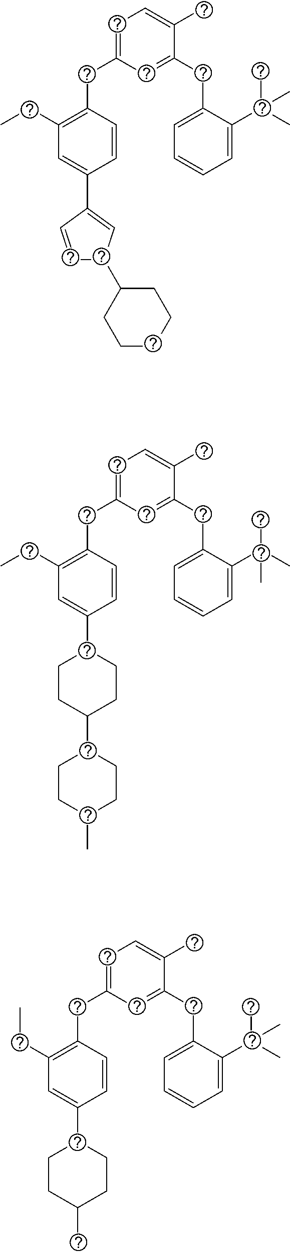

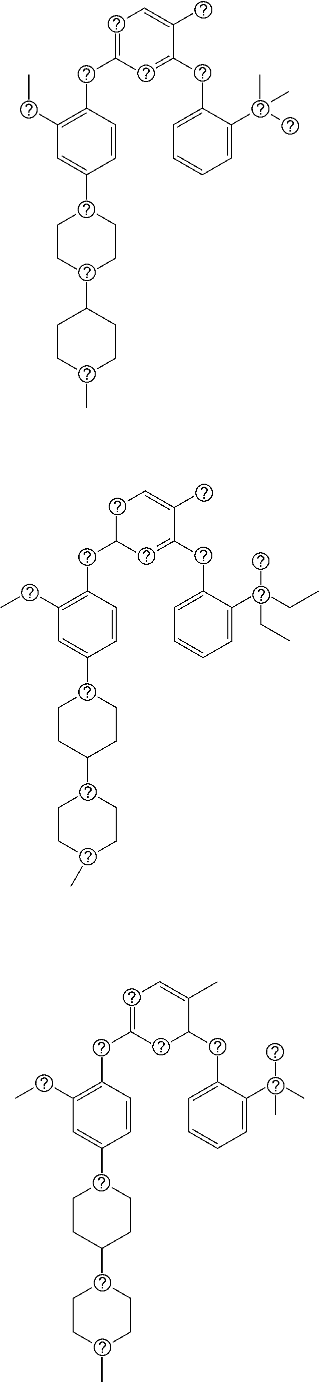

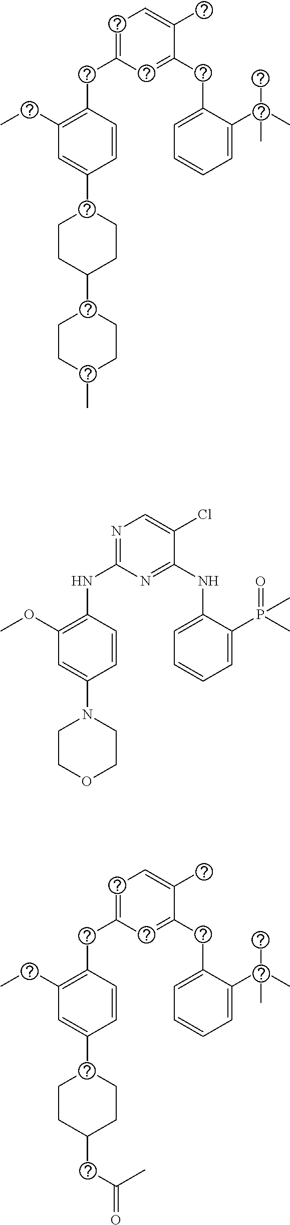

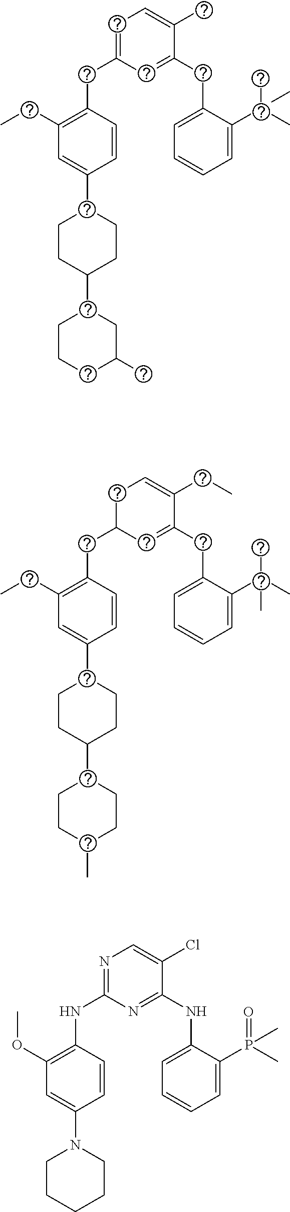

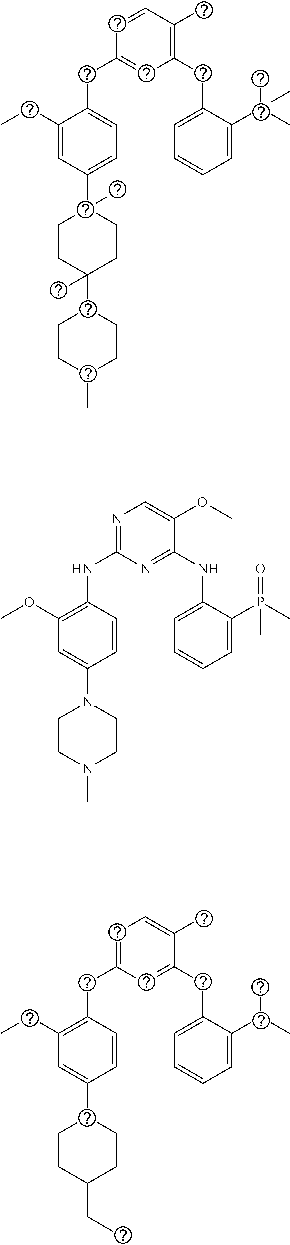

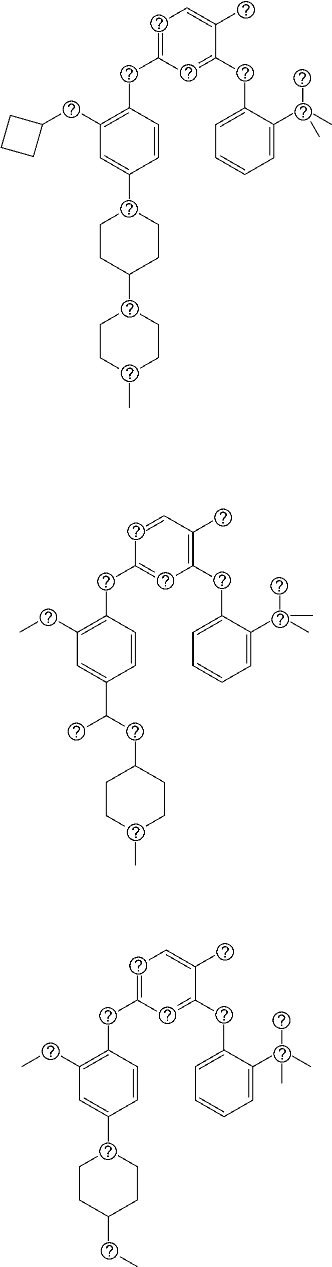

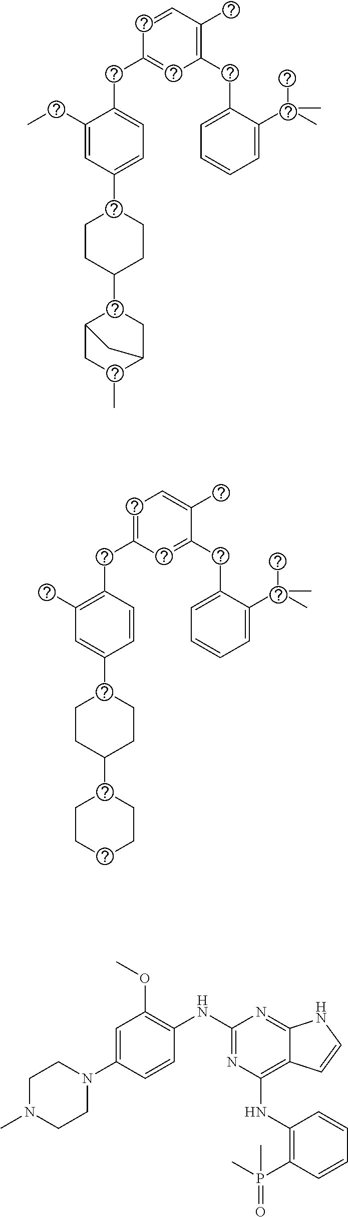

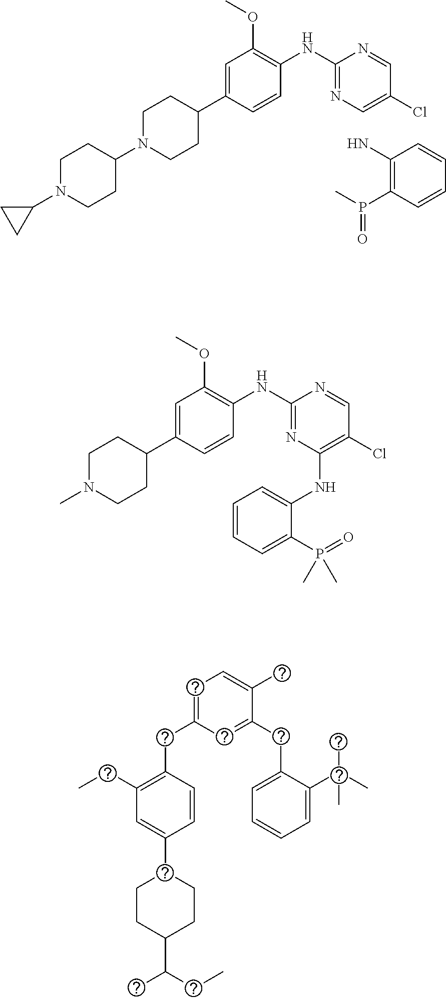

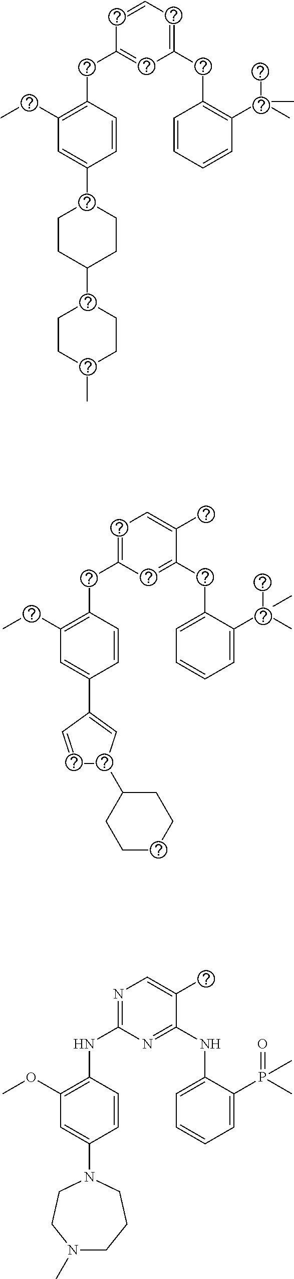

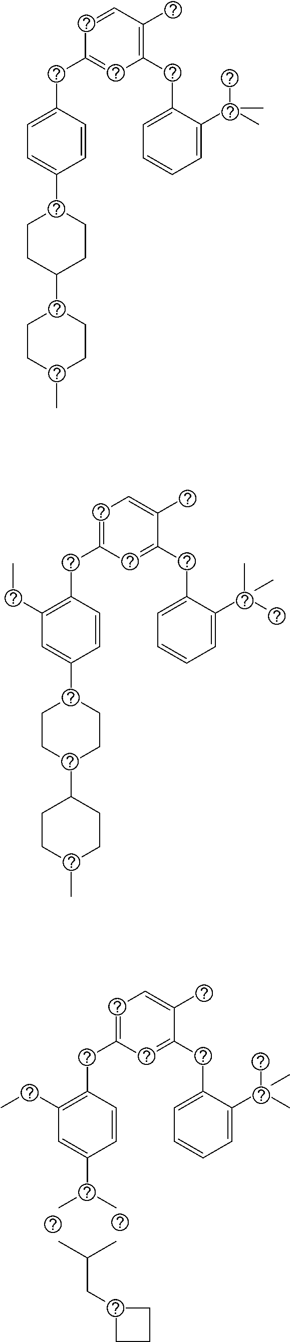

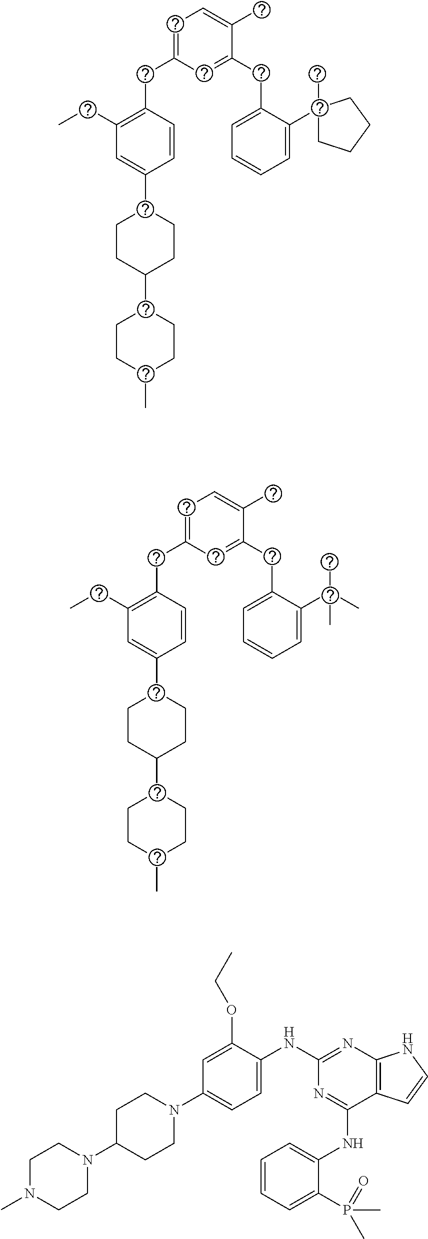

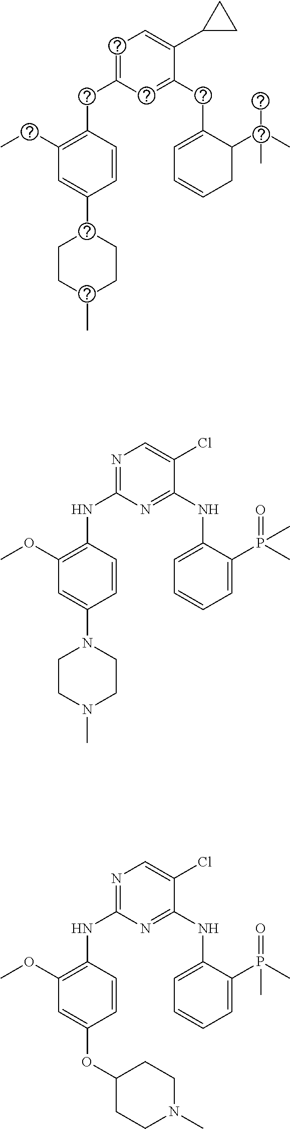

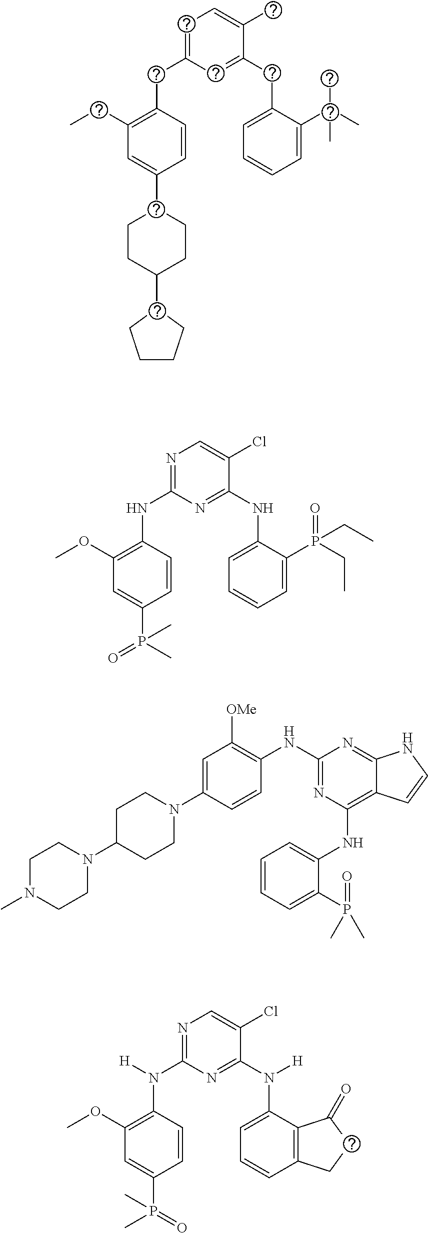

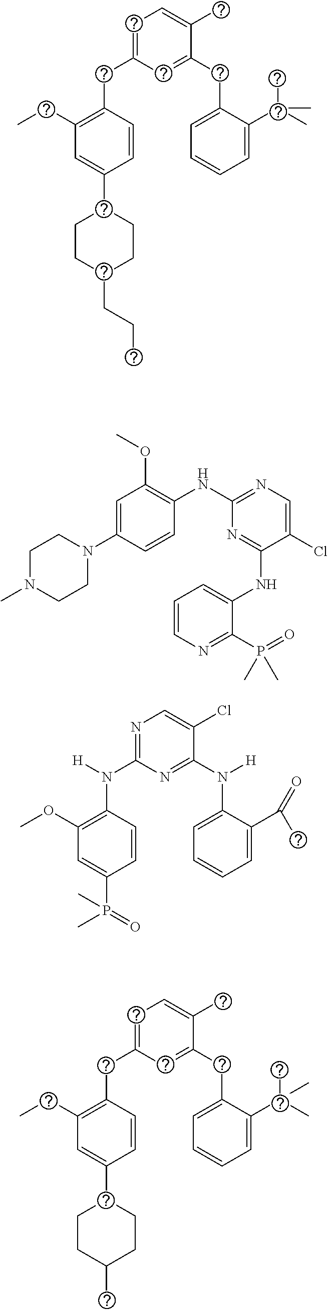

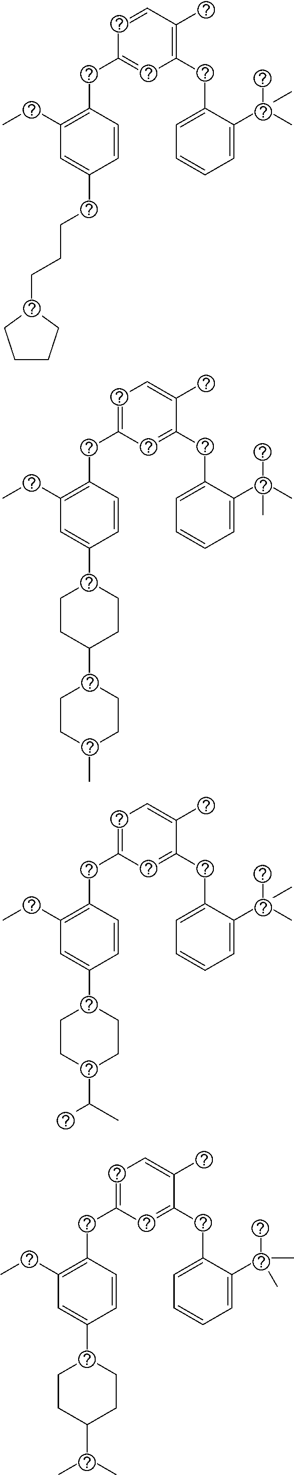

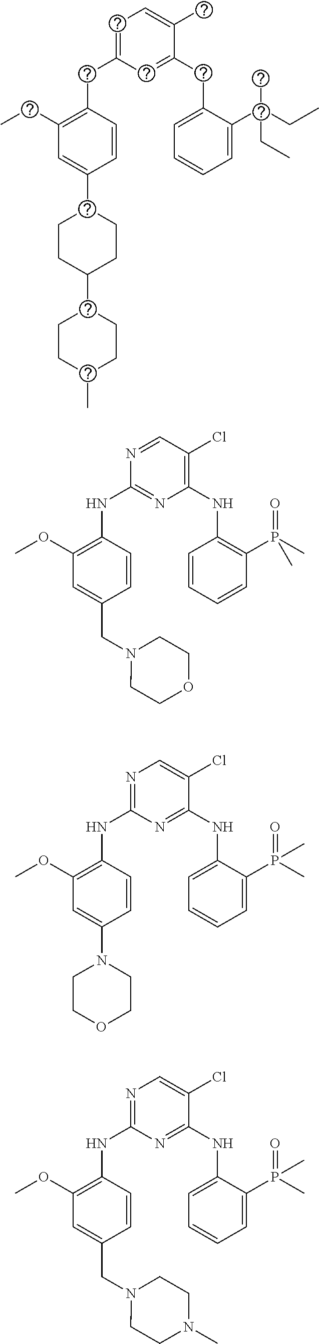

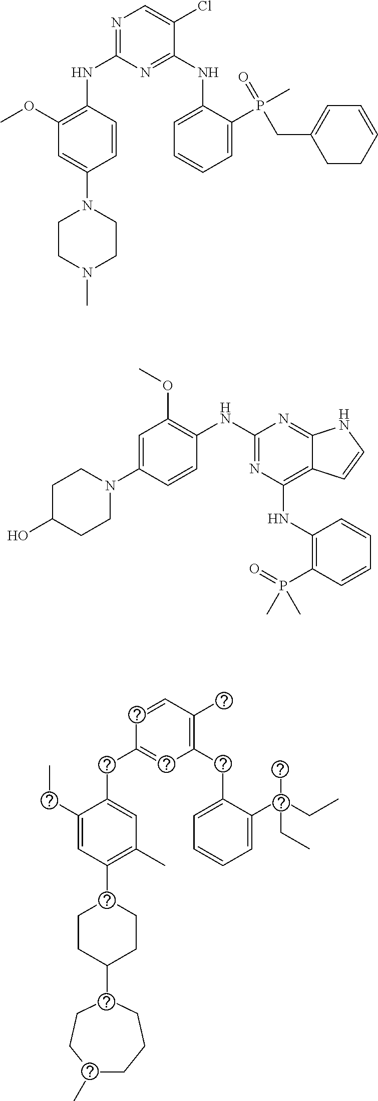

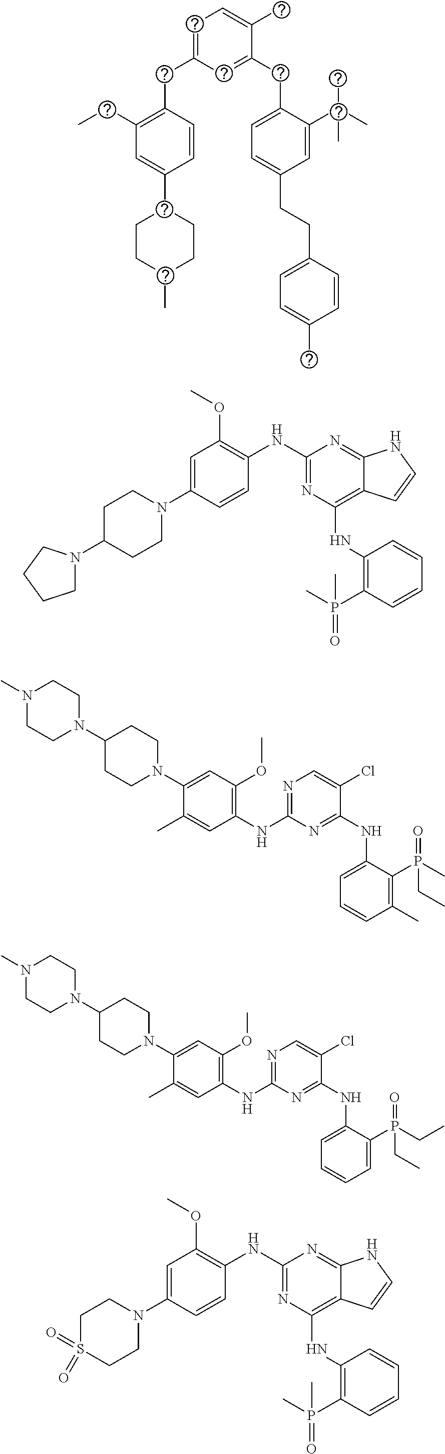

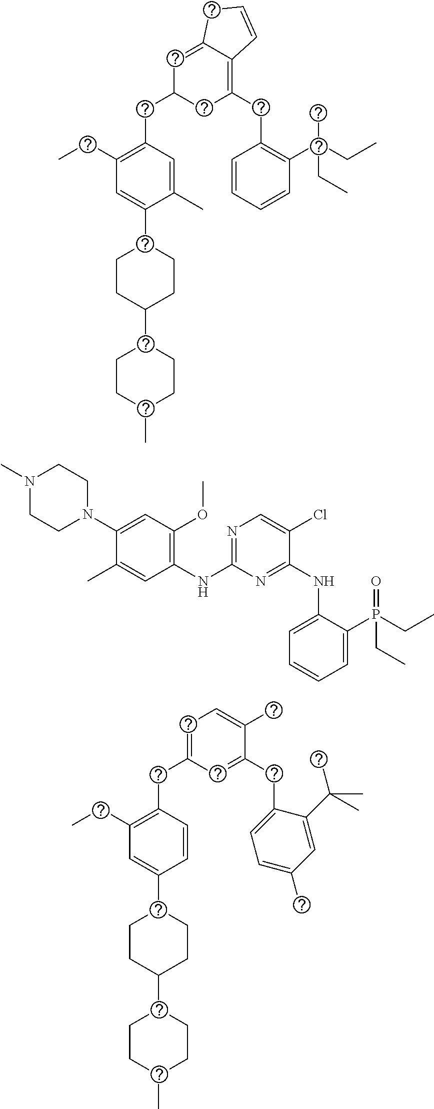

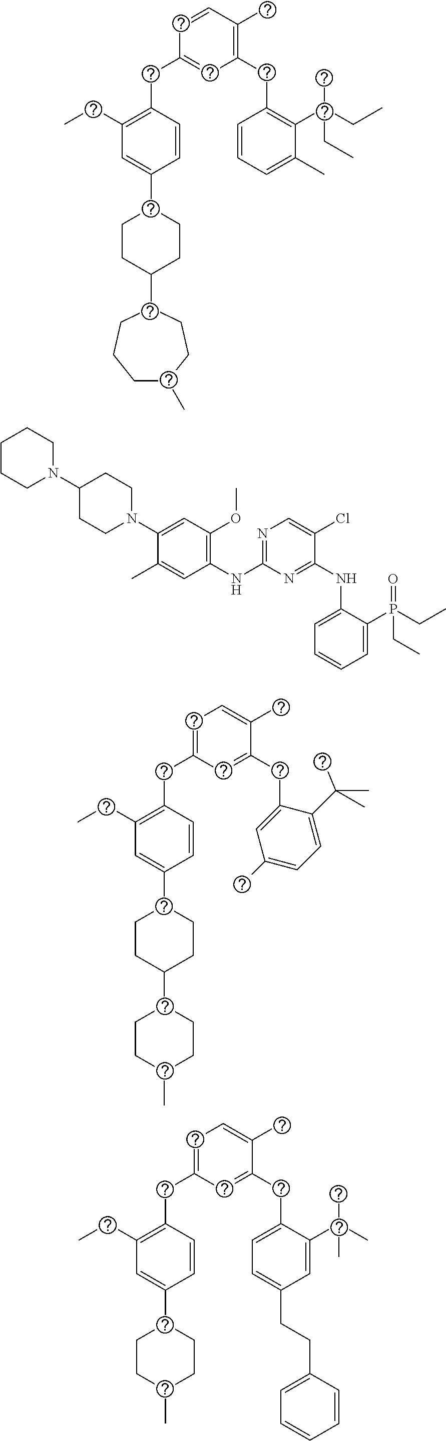

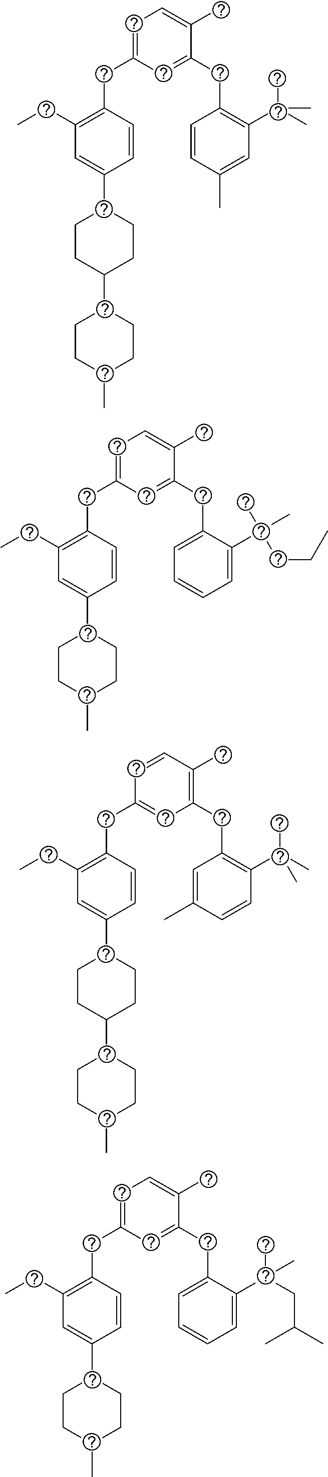

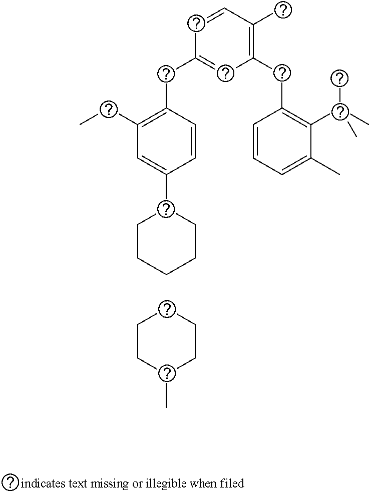

### Example 5

### NSCLC Models for EGFR-T790M

The compounds of formula (I) can further be tested using a model for NSCLC using cell lines HCC827(EGFR Del E746_A750) or H1975 (EGFR L858R/T790M). These cell lines were used as models used in second generation EGFR-I development. Pharmacokinetics/pharmacodynamics (PK/PD) and efficacy studies can also be conducted, using, for example, BIBW 2992 as a reference compound.

### Example 6

### Clinical Administration

The compound of formula (I) can be formulated for oral delivery using conventional methods and materials, including loading of compound into capsules with or without conventional excipients.

Dosing in a first human clinical trial began at an oral dose level of 30 mg per day using capsules containing a compound of formula (I) without excipients. That starting dose was chosen based on the ADME, pharmacokinetic and toxicity studies of the compound and is expected to be followed by 60 mg, 90 mg, 120 mg and higher daily doses.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for treating an EGFR-driven cancer in a subject, the method comprising administering to the subject a therapeutically effective amount of a compound of formula (I), or a pharmaceutically acceptable salt thereof: 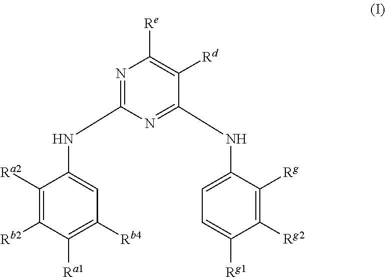
wherein
R^{d} is H, C₁₋₄ alkyl, C₁₋₄ alkoxy, or halo; and R^{e} is H or NH₂; or R^{d} and R^{e}, together with the pyrimidine ring atoms to which they are attached, form a 5- or 6-membered ring containing 1, 2 or 3 heteroatoms, independently selected from N, S and O, wherein the 5- or 6-membered ring is substituted by R^{h};
R^{h} is H, C₁₋₄ alkyl, or halo;
R^{a2} is H, C₁₋₆ alkoxy, C₃₋₆ alkenyloxy, or C₃₋₆ cycloalkyloxy;
R^{g} is —P(O)(R^{3A})(R^{3B}), —S(O)N(R^{3C})(R^{3D}), —S(O)₂R^{3E}, —OC(O)N(R^{3F})(R^{3G}), —NR^{3H}C(O)OR^{3I}, a 5 or 6 member heterocyclic ring comprising 1, 2, 3 or 4 N atoms, or combined with R^{g2} forms a 5- to 7-member heterocyclic ring, wherein each of R^{3A}, R^{3B}, R^{3C}, R^{3D}, R^{3E}, R^{3F}, R^{3G}, R^{3H}, and R^{3I} is, independently, selected from H, alkyl, alkenyl, alkynyl, cycloalkyl, cycloalkenyl, cycloalkynyl, and heteroalkyl, or R^{3A} and R^{3B}, or R^{3C} and R^{3D}, or R^{3F} and R^{3G}, together with the atoms to which they are attached, combine to form a 5- or 6-membered heterocyclic ring which is unsubstituted or substituted;
R^{g2} is H, F, C₁₋₄ alkyl, or, R^{g2} and R^{g} together with the atoms to which they are attached form a 5- to 7-member heterocyclic ring comprising 1-3 hetero atoms independently selected from P, N, O and S, the heterocyclic ring being unsubstituted or substituted;
R^{g1} is H, F, or a 5 or 6 member heterocyclic ring comprising 1 or 2 N atoms, the heterocyclic ring being unsubstituted or substituted;
R^{b2} is H, F, or is a 5 or 6 member heterocyclic ring containing 1, 2 or 3 N or O atoms, the heterocyclic ring being unsubstituted or substituted;
R^{b4} is H, F, C₁₋₆ alkoxy, C₃₋₆ alkenyloxy, or C₃₋₆ cycloalkyloxy, —OC(O)N(R^{5A})(R^{5B}), —NR^{5C}C(O)OR^{5D}; a 5 or 6 member heterocyclic ring comprising 1, 2 or 3 N or O atoms, the heterocyclic ring being unsubstituted or substituted, or, R^{b4} and R^{a1} together with the atoms to which they are attached form a 6 member heterocyclic ring comprising 1, 2 or 3 N or O atoms which is unsubstituted or substituted;
each of R^{5A}, R^{5B}, R^{5C}, and R^{5D} is, independently, selected from H, alkyl, alkenyl, alkynyl, and heteroalkyl, or R^{5A} and R^{5B}, together with the atoms to which they are attached, combine to form a 5- or 6-membered heterocyclic ring which is unsubstituted or substituted;
R^{a1} combines with R^{b4} to form a 6 member heterocyclic ring, or R^{a1} is H, halo, —CN,
—NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or 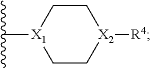
each Y is, independently, a bond, —O—, —S— or —NR¹—;
each occurrence of R¹ and R² is, independently H, alkyl, alkenyl, alkynyl, cycloalkyl, cycloalkenyl, cycloalkynyl, aryl, heteroalkyl, heterocyclic or heteroaryl;
each of X₁ and X₂ is, independently, CH or N; and
R⁴ is alkyl, alkenyl, alkynyl, cycloalkyl, cycloalkenyl, cycloalkynyl, aryl, heteroalkyl, heterocyclic or heteroaryl.

2. A method for treating an EGFR-driven cancer in a subject:
a) providing a subject having a cancer characterized by the presence of a mutation in epidermal growth factor receptor kinase (EGFR), and
b) administering to the subject a therapeutically effective amount of compound of formula (I), or a pharmaceutically acceptable salt thereof: 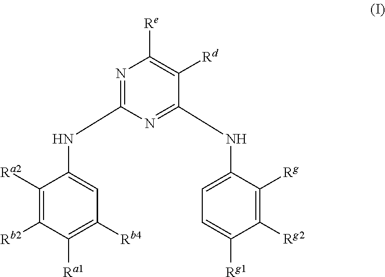
wherein
R^{d} is H, C₁₋₄ alkyl, C₁₋₄ alkoxy, or halo; and R^{e} is H or NH₂; or R^{d} and R^{e}, together with the pyrimidine ring atoms to which they are attached, form a 5- or 6-membered ring containing 1, 2 or 3 heteroatoms, independently selected from N, S and O, wherein the 5- or 6-membered ring is substituted by R^{h};
R^{h} is H, C₁₋₄ alkyl, or halo;
R^{a2} is H, C₁₋₆ alkoxy, C₃₋₆ alkenyloxy, or C₃₋₆ cycloalkyloxy;
R^{g} is —P(O)(R^{3A})(R^{3B}), —S(O)N(R^{3C})(R^{3D}), —S(O)₂R^{3E}, —OC(O)N(R^{3F})(R^{3G}), —NR^{3H}C(O)OR^{3I}, a 5 or 6 member heterocyclic ring comprising 1, 2, 3 or 4 N atoms, or combined with R^{g2} forms a 5- to 7-member heterocyclic ring, wherein each of R^{3A}, R^{3B}, R^{3C}, R^{3D}, R^{3E}, R^{3F}, R^{3G}, R^{3H}, and R^{3I} is, independently, selected from H, alkyl, alkenyl, alkynyl, cycloalkyl, cycloalkenyl, cycloalkynyl, and heteroalkyl, or R^{3A} and R^{3B}, or R^{3C} and R^{3D}, or R^{3F} and R^{3G}, together with the atoms to which they are attached, combine to form a 5- or 6-membered heterocyclic ring which is unsubstituted or substituted;
R^{g2} is H, F, C₁₋₄ alkyl, or, R^{g2} and R^{g} together with the atoms to which they are attached form a 5- to 7-member heterocyclic ring comprising 1-3 hetero atoms independently selected from P, N, O and S, the heterocyclic ring being unsubstituted or substituted;
R^{g1} is H, F, or a 5 or 6 member heterocyclic ring comprising 1 or 2 N atoms, the heterocyclic ring being unsubstituted or substituted;
R^{b2} is H, F, or is a 5 or 6 member heterocyclic ring containing 1, 2 or 3 N or O atoms, the heterocyclic ring being unsubstituted or substituted;
R^{b4} is H, F, C₁₋₆ alkoxy, C₃₋₆ alkenyloxy, or C₃₋₆ cycloalkyloxy, —OC(O)N(R^{5A})(R^{5B}), —NR^{5C}(O)OR^{5D}; a 5 or 6 member heterocyclic ring comprising 1, 2 or 3 N or O atoms, the heterocyclic ring being unsubstituted or substituted, or, R^{b4} and R^{a1} together with the atoms to which they are attached form a 6 member heterocyclic ring comprising 1, 2 or 3 N or O atoms which is unsubstituted or substituted;
each of R^{5A}, R^{5B}, R^{5C}, and R^{5D} is, independently, H, alkyl, alkenyl, alkynyl, or heteroalkyl; or R^{5A} and R^{5B}, together with the atoms to which they are attached, combine to form a 5- or 6-membered heterocyclic ring which is unsubstituted or substituted;
R^{a1} combines with R^{b4} to form a 6 member heterocyclic ring, or is H, halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or 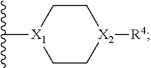
each Y is, independently, a bond, —O—, —S— or —NR¹—;
each occurrence of R¹ and R² is, independently, H, alkyl, alkenyl, alkynyl, cycloalkyl, cycloalkenyl, cycloalkynyl, aryl, heteroalkyl, heterocyclic or heteroaryl;
each of X₁ and X₂ is, independently, CH or N; and
R⁴ is alkyl, alkenyl, alkynyl, cycloalkyl, cycloalkenyl, cycloalkynyl, aryl, heteroalkyl, heterocyclic or heteroaryl.

3. The method of claim 2, wherein the EGFR-driven cancer is characterized by the presence of one or more mutations selected from: (i) L858R, (ii) T790M, (iii) both L858R and T790M, (iv) delE746_A750, and (v) both delE746_A750 and T790M.

4. The method of any of claims 1-3, wherein the EGFR-driven cancer is a non-small cell lung cancer (NSCLS); glioblastoma; pancreatic cancer; head and neck cancer (e.g., squamous cell carcinoma); breast cancer; colorectal cancer; epithelial cancer; ovarian cancer; prostate cancer; or an adenocarcinoma.

5. A method of inhibiting the proliferation of a cell expressing an EGFR mutant, the method comprising contacting the cell with a compound of formula (I), or a pharmaceutically acceptable salt thereof: 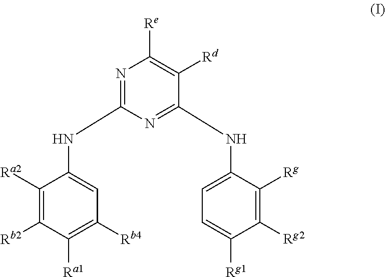
wherein
R^{d} is H, C1-4 alkyl, C1-4 alkoxy, or halo; and R^{e} is H or NH₂; or R^{d} and R^{e}, together with the pyrimidine ring atoms to which they are attached, form a 5- or 6-membered ring containing 1 or 2 heteroatoms, independently selected from N, S and O, wherein the 5- or 6-membered ring is substituted by R^{h};
R^{h} is H, C₁₋₄ alkyl, or halo;
R^{a2} is H, C₁₋₆ alkoxy, C₃₋₆ alkenyloxy, or C₃₋₆ cycloalkyloxy;
R^{g} is —P(O)(R^{3A})(R^{3B}), —S(O)N(R^{3C})(R^{3D}), —S(O)₂R^{3E}, —OC(O)N(R^{3F})(R^{3G}), —NR^{3H}C(O)OR^{3I}, a 5 or 6 member heterocyclic ring comprising 1, 2, 3 or 4 N atoms, or combined with R^{g2} forms a 5- to 7-member heterocyclic ring, wherein each of R^{3A}, R^{3B}, R^{3C}, R^{3D}, R^{3E}, R^{3F}, R^{3G}, R^{3H}, and R^{3I} is, independently, selected from H, alkyl, alkenyl, alkynyl, cycloalkyl, cycloalkenyl, cycloalkynyl, and heteroalkyl, or R^{3A} and R^{3B}, or R^{3C} and R^{3D}, or R^{3F} and R^{3G}, together with the atoms to which they are attached, combine to form a 5- or 6-membered heterocyclic ring which is unsubstituted or substituted;
R^{g2} is H, F, 014 alkyl, or, R^{g2} and R^{g} together with the atoms to which they are attached form a 5- to 7-member heterocyclic ring comprising 1-3 hetero atoms independently selected from P, N, O and S, the heterocyclic ring being unsubstituted or substituted;
R^{g1} is H, F, or a 5 or 6 member heterocyclic ring comprising 1 or 2 N atoms, the heterocyclic ring being unsubstituted or substituted;
R^{b2} is H, F, or is a 5 or 6 member heterocyclic ring containing 1, 2 or 3 N or O atoms, the heterocyclic ring being unsubstituted or substituted;
R^{b4} is H, F, C₁₋₆ alkoxy, C₃₋₆ alkenyloxy, or C₃₋₆ cycloalkyloxy, —OC(O)N(R^{5A})(R^{5B}), —NR^{5C}C(O)OR^{5D}; a 5 or 6 member heterocyclic ring comprising 1, 2 or 3 N or O atoms, the heterocyclic ring being unsubstituted or substituted, or, R^{b4} and R^{a1} together with the atoms to which they are attached form a 6 member heterocyclic ring comprising 1, 2 or 3 N or O atoms which is unsubstituted or substituted;
each of R^{5A}, R^{5B}, R^{5C}, and R^{5D} is, independently, selected from H, alkyl, alkenyl, alkynyl, and heteroalkyl, or R^{5A} and R^{5B}, together with the atoms to which they are attached, combine to form a 5- or 6-membered heterocyclic ring which is unsubstituted or substituted;
R^{a1} combines with R^{b4} to form a 6 member heterocyclic ring, or is H, halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or 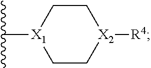
each Y is, independently, a bond, —O—, —S— or —NR¹—;
each occurrence of R¹ and R² is, independently, H, alkyl, alkenyl, alkynyl, cycloalkyl, cycloalkenyl, cycloalkynyl, aryl, heteroalkyl, heterocyclic or heteroaryl;
each of X₁ and X₂ is, independently, CH or N; and
R⁴ is alkyl, alkenyl, alkynyl, cycloalkyl, cycloalkenyl, cycloalkynyl, aryl, heteroalkyl, heterocyclic or heteroaryl,
in an amount sufficient to inhibit the proliferation.

6. The method of claim 5, wherein the EGFR mutant is characterized by the presence of one or more mutations in epidermal growth factor receptor kinase (EGFR) selected from: (i) L858R, (ii) T790M, (iii) both L858R and T790M, (iv) delE746_A750, and (v) both delE746_A750 and T790M.

7. The method of claim 5 or 6, wherein the cell is a cancer cell.

8. The method of claims 1-3, wherein the cancer cell is a cell from a non-small cell lung cancer (NSCLS); glioblastoma; pancreatic cancer; head and neck cancer (e.g., squamous cell carcinoma); breast cancer; colorectal cancer; epithelial cancer; ovarian cancer; prostate cancer; or an adenocarcinoma.

9. A method of treating an EGFR-driven cancer refractory to erlotinib or gefitinib, or to a pharmaceutically acceptable salt of either, in a subject, the method comprising administering to the subject a compound of formula I: 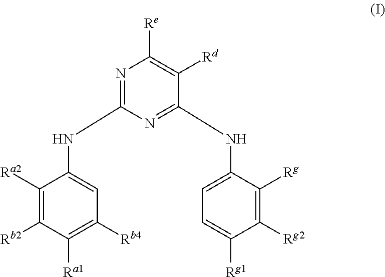
wherein
R^{d} is H, C₁₋₄ alkyl, C₁₋₄ alkoxy, or halo; and R^{e} is H or NH₂; or R^{d} and R^{e}, together with the pyrimidine ring atoms to which they are attached, form a 5- or 6-membered ring containing 1, 2, or 3 heteroatoms, independently selected from N, S and O, wherein the 5- or 6-membered ring is substituted by R^{h};
R^{h} is H, C₁₋₄ alkyl, or halo;
R^{a2} is H, C₁₋₆ alkoxy, C₃₋₆ alkenyloxy, or C₃₋₆ cycloalkyloxy;
R^{g} is —P(O)(R^{3A})(R^{3B}), —S(O)N(R^{3C})(R^{3D}), —S(O)₂R^{3E}, —OC(O)N(R^{3F})(R^{3G}), —NR^{3H}C(O)OR^{3I}, a 5 or 6 member heterocyclic ring comprising 1, 2, 3 or 4 N atoms, or combined with R^{g2} forms a 5- to 7-member heterocyclic ring, wherein each of R^{3A}, R^{3B}, R^{3C}, R^{3D}, R^{3E}, R^{3F}, R^{3G}, R^{3H}, and R^{3I} is, independently, selected from H, alkyl, alkenyl, alkynyl, cycloalkyl, cycloalkenyl, cycloalkynyl, and heteroalkyl, or R^{3A} and R^{3B}, or R^{3C} and R^{3D}, or R^{3F} and R^{3G}, together with the atoms to which they are attached, combine to form a 5- or 6-membered heterocyclic ring which is unsubstituted or substituted;
R^{g2} is H, F, C₁₋₄ alkyl, or, R^{g2} and R^{g} together with the atoms to which they are attached form a 5- to 7-member heterocyclic ring comprising 1-3 hetero atoms independently selected from P, N, O and S, the heterocyclic ring being unsubstituted or substituted;
R^{g1} is H, F, or a 5 or 6 member heterocyclic ring comprising 1 or 2 N atoms, the heterocyclic ring being unsubstituted or substituted;
R^{b2} is H, F, or is a 5 or 6 member heterocyclic ring containing 1, 2 or 3 N or O atoms, the heterocyclic ring being unsubstituted or substituted;
R^{b4} is H, F, C₁₋₆ alkoxy, C₃₋₆ alkenyloxy, or C₃₋₆ cycloalkyloxy, —OC(O)N(R^{5A})(R^{5B}), —NR^{5C}C(O)OR^{5D}; a 5 or 6 member heterocyclic ring comprising 1, 2 or 3 N or O atoms, the heterocyclic ring being unsubstituted or substituted, or, R^{b4} and R^{a1} together with the atoms to which they are attached form a 6 member heterocyclic ring comprising 1, 2 or 3 N or O atoms which is unsubstituted or substituted;
each of R^{5A}, R^{5B}, R^{BC}, and R^{5D} is, independently, selected from H, alkyl, alkenyl, alkynyl, and heteroalkyl, or R^{5A} and R^{5B}, together with the atoms to which they are attached, combine to form a 5- or 6-membered heterocyclic ring which is unsubstituted or substituted;
R^{a1} combines with R^{b4} to form a 6 member heterocyclic ring, or is H, halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or 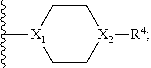
each Y is, independently, a bond, —O—, —S— or —NR¹—;
each occurrence of R¹ and R² is, independently, H, alkyl, alkenyl, alkynyl, cycloalkyl, cycloalkenyl, cycloalkynyl, aryl, heteroalkyl, heterocyclic or heteroaryl;
each of X₁ and X₂ is, independently, CH or N; and
R⁴ is alkyl, alkenyl, alkynyl, cycloalkyl, cycloalkenyl, cycloalkynyl, aryl, heteroalkyl, heterocyclic or heteroaryl, or a pharmaceutically acceptable salt thereof, in an amount sufficient to treat the cancer.

10. The method of any of claims 1-9, wherein the compound of formula (I) is described by formula (IIa) or formula (IIb), or a pharmaceutically acceptable salt thereof: 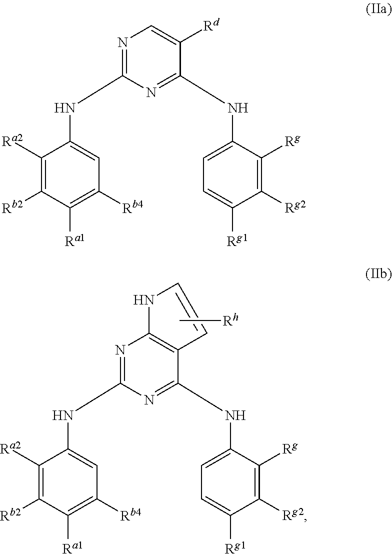
wherein R^{a1}; R^{b2}; R^{b4}; R^{g}; R^{g1}; R^{g2}; R^{d}; and R^{h} are as defined in formula (I).

11. The method of claim 10, wherein R^{g1}, R^{g2}, R^{b2} and Ware H or F.

12. The method of claim 10, wherein R^{d} is Cl, F or CF₃.

13. The method of claim 10, wherein R^{a1} is methoxy.

14. The method of claim 10, wherein R^{g} is —P(O)(R^{3A})(R^{3B}) or —S(O)₂R^{3E}, wherein R^{3A}; R^{3B}; and R^{3E} are as defined in formula (I).

15. The method of claim 10, wherein R^{a1} is a 5 or 6 member heterocyclic ring comprising one or two N or O atoms and which is unsubstituted or substituted with an alkyl group.

16. The method of any of claims 1-9, wherein the compound of formula (I) is described by formula (III) or a pharmaceutically acceptable salt thereof: 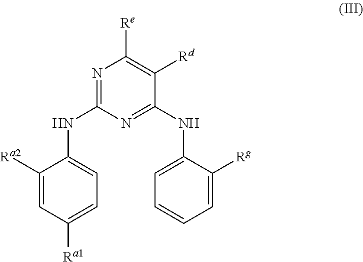
wherein
R^{a2} is alkoxy;
R^{g} is —P(O)(R^{3A})(R^{3B}); —S(O)N(R^{3C})(R^{3D}); or —S(O)₂R^{3E};
each of R^{3A}, R^{3B}, R^{3C}, R^{3D}, and R^{3E} is, independently, selected from H and C₁₋₇ alkyl, or R^{3A} and R^{3B}, or R^{3C} and R^{3D}, together with the atoms to which they are attached, combine to form a 5- or 6-membered heterocyclic ring which is unsubstituted or substituted;
R^{d} is H, C₁₋₄ alkyl, C₁₋₄ alkoxy, or halo; and R^{e} is H or NH₂; or R^{d} and R^{e}, together with the pyrimidine ring atoms to which they are attached, form a 5- or 6-membered ring containing 1 or 2 heteroatoms, independently selected from N, S and O, wherein the 5- or 6-membered ring is substituted by R^{h};
R^{h} is H, C₁₋₄ alkyl, or halo;
R^{a1} is halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR¹)(YR²), —NR¹SO₂R², —S(O)ᵣR², —SO₂NR¹R², —NR¹SO₂NR¹R², or 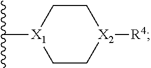
each Y is, independently, a bond, —O—, —S— or —NR¹—;
each occurrence of R¹ and R² is, independently, selected from H, alkyl, alkenyl, alkynyl, cycloalkyl, cycloalkenyl, cycloalkynyl, aryl, heteroalkyl, heterocyclic and heteroaryl;
each of X₁ and X₂ is, independently, selected from CH and N; and
R⁴ is selected from alkyl, alkenyl, alkynyl, cycloalkyl, cycloalkenyl, cycloalkynyl, aryl, heteroalkyl, heterocyclic and heteroaryl.

17. The method of claim 16, wherein the compound of formula (III) is described by formula (IVa) or formula (IVb) or a pharmaceutically acceptable salt thereof: 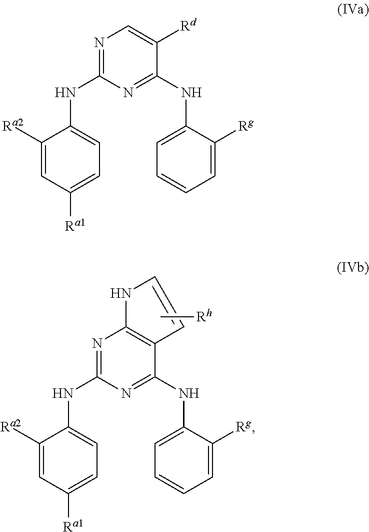
wherein R^{a2}; R^{g}; R^{d}; R^{h}; and R^{a1} are as defined in formula (III).

18. The method of claim 16, wherein R^{a2} is a methoxy, ethoxy, or propoxy group.

19. The method of claim 17, wherein R^{d} is Cl, F, CF₃, or cyclopropyl.

20. The method of claim 16, wherein the compound of formula (III) is described by any of formulas (Va)-(Vd) or a pharmaceutically acceptable salt thereof: 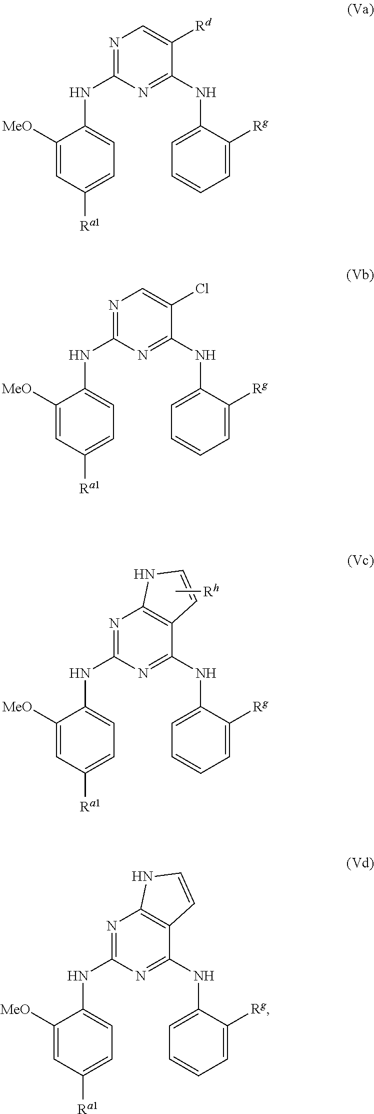
wherein R^{g}; R^{d}; R^{h}; and R^{a1} are as defined in formula (III).

21. The method of claim 20, wherein R^{g} is —P(O)(CH₃)₂ or —S(O)₂(CH(CH₃)₂).

22. The method of claim 20, wherein R^{a1} is: 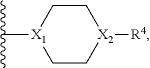
wherein X₁, X₂, and R⁴ are as defined in formula (III).

23. The method of claim 22, wherein R^{a1} is selected from any of the following groups:

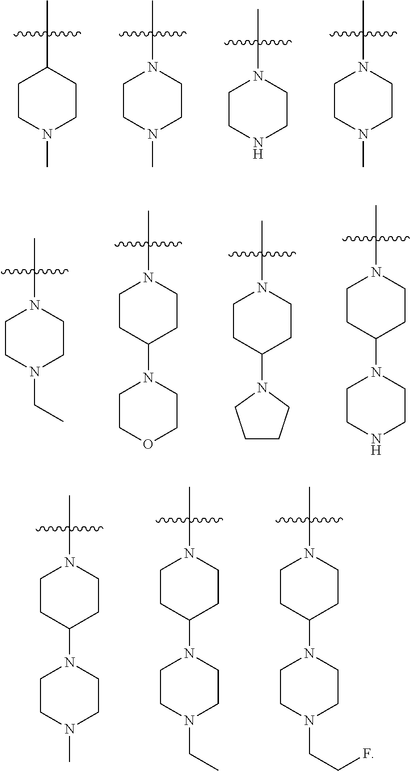

24. The method of claim 16, wherein the compound of formula (III) is described by any of formulas (VIa)-(VIf) or a pharmaceutically acceptable salt thereof: 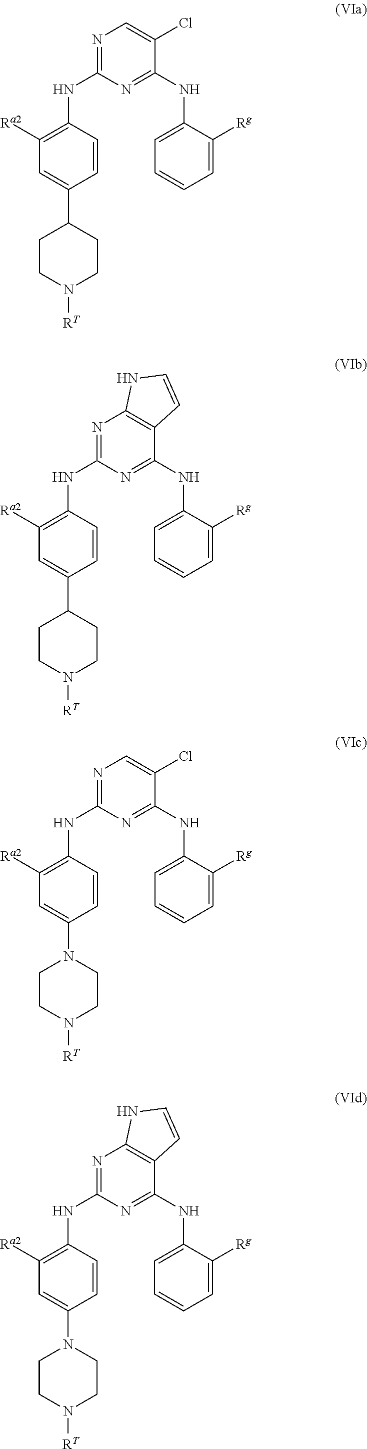 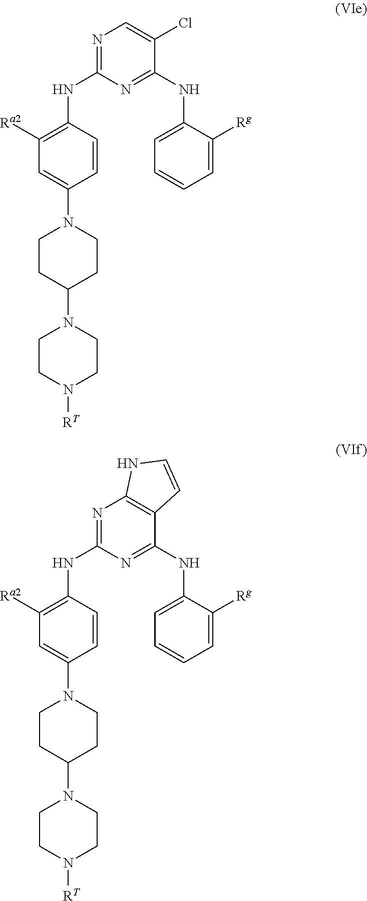
wherein
R^{a2} is a methoxy, ethoxy, or propoxy group;
R^{g} is —P(O)(CH₃)₂, —P(O)(CH₂CH₃)₂, or —S(O)₂(CH(CH₃)₂); and,
R^{T} is H, acyl or C₁-C₄ alkyl, which may be substituted or unsubstituted.
